(19) United States
(12) Patent Application Publication
(10) Pub. No.: US 2007/0249686 A1
(43) Pub. Date: Oct. 25, 2007

(54) Modulators of Crth-2 Receptor Activity for the Treatment of Prostaglandin D2 Mediated Diseases

(75) Inventors: Roger Victor Bonnert, Leicestershire (GB); Timothy Jon Luker, Leicestershire (GB); Garry Pairaudeau, Leicestershire (GB); Stephen Thom, Leicestershire (GB)

Correspondence Address: FISH & RICHARDSON P.C., P.O BOX 1022, MINNEAPOLIS, MN, 55440-1022, US

(73) Assignee: ASTRAZENECA AB, Södertälje (SE)

(21) Appl. No.: 11/576,372

(22) PCT Filed: Oct. 3, 2005
(86) PCT No.: PCT/GB05/03794

§ 371 Date: Mar. 30, 2007

(30) Foreign Application Priority Data
Oct. 5, 2004 (GB) 0422057.0

(51) Int. Cl. A61K 31/44; A61K 31/19; A61K 31/34; A61K 31/38; A61P 11/00; C07D 209/08; C07D 213/73; C07D 261/08; C07D 307/42; C07D 333/38; C07D 333/28; C07D 333/22; C07D 333/16; C07D 295/18; C07D 231/12; C07D 213/65; C07C 57/30; A61K 31/40; A61K 31/415; A61K 31/42

(52) U.S. Cl. 514/345; 514/349; 514/378; 514/406; 514/418; 514/423; 514/438; 514/447; 514/461; 514/568; 546/290; 546/297; 548/247; 548/376.1; 548/491; 548/530; 549/499; 549/61; 549/76; 562/405; 562/426

(57) ABSTRACT

The invention relates to substituted acids as useful pharmaceutical compounds for treating respiratory disorders as asthma, pharmaceutical compositions containing them, and processes for their preparation.

The present invention relates to substituted acids as useful pharmaceutical compounds for treating respiratory disorders, pharmaceutical compositions containing them, and processes for their preparation.

EPA 1 170 594 discloses methods for the identification of compounds useful for the treatment of disease states mediated by prostaglandin D2, a ligand for orphan receptor CRTH2. GB 1356834 discloses a series of compounds said to possess anti-inflammatory, analgesic and antipyretic activity. It has been found that certain acids are active at the CRTH2 receptor, and as a consequence are expected to be potentially useful for the treatment of various respiratory diseases, including asthma and COPD.

In a first aspect the invention therefore provides a compound of formula (I) or a pharmaceutically acceptable salt or solvate thereof: 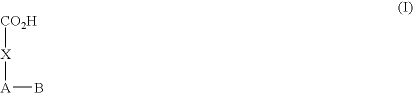
in which:
X is YCR¹R² or CR³═CR⁴,

A is aryl or heteroaryl, optionally substituted by one or more substituents independently selected from hydrogen, halogen, CN, OH, SH, nitro, S(O)ₙR⁵ (where n is 0, 1 or 2), OR⁵, NR⁶R⁷ or C₁₋₆alkyl, the latter group being optionally substituted by one or more halogen atoms.

B is aryl or heteroaryl, optionally substituted by one or more substituents independently selected from from hydrogen, halogen, CN, OH, SH, nitro, CO₂R⁶, COR⁶, SO₂R⁸, OR⁸, SR⁸, SOR⁸, SO₂NR⁹R¹⁰, CONR⁹R¹⁰, NR⁹R¹⁰, NHSO₂R⁸, NR⁸SO₂R⁸, NR⁸CO₂R⁸, NHCOR⁸, NR⁸COR⁸, NR⁶CONR⁶R⁷, NR⁶SO₂NR⁶R⁷, aryl, heteroaryl, C₂-C₆ alkenyl, C₂-C₆ alkynyl, C₃-C₇ cycloalkyl or C₁-6alkyl, the latter four groups being optionally substituted by one or more substituents independently selected from halogen, C₃-C₇ cycloalkyl, OR⁶, NR⁶R⁷, S(O)ₙR⁵ (where n is 0, 1 or 2), CONR⁶R⁷, NR⁶COR⁷, SO₂NR⁶R⁷ and NR⁶SO₂R⁵;

X and B are attached to the the aryl or heteroaryl ring ortho relative to each other

Y is a bond, O, S(O)ₙ (where n is 0, 1 or 2), NR³ or CR¹R²;

R¹ and R² independently represent a hydrogen atom, halogen, C₂-C₆ alkenyl, C₂-C₆ alkynyl, C₃-C₇ cycloalkyl or a C₁₋₆alkyl group, the latter four groups being optionally substituted by one or more substituents independently selected from halogen, C₃-C₇ cycloalkyl, NR³R⁴, OR³, S(O)ₙR⁵ (where n is 0, 1 or 2); or

R¹ and R² together can form a 3-8 membered ring optionally containing one or more atoms selected from O, S, NR¹¹ and itself optionally substituted by one or more C₁-C₃ alkyl or halogen;

R³ and R⁴ independently represent hydrogen, or C₁₋₆alkyl

R⁵ is C₁₋₆alkyl or C₃-C₇ cycloalkyl, optionally substituted by one or more halogen atoms

R⁶ and R⁷ independently represents a hydrogen atom, C₁-C₆ alkyl or C₃-C₇ cycloalkyl, optionally substituted by one or more halogen atoms

R⁸ represents aryl, heteroaryl, C₃-C₇ cycloalkyl or C₁₋₆alkyl, the latter two groups may be optionally substituted by one or more substituents independently selected from halogen, C₃-C₇ cycloalkyl, aryl, heteroaryl OR⁶ and NR⁶R⁷, S(O)ₙR⁵ (where n=0, 1 or 2), CONR⁶R⁷, NR⁶COR⁷, SO₂NR⁶R⁷ and NR⁶SO₂R⁵;

R⁹ and R¹⁰ independently represent aryl or heteroaryl, hydrogen, C₃-C₇ cycloalkyl or C₁₋₆alkyl, the latter two groups being optionally substituted by one or more substituents independently selected from halogen, C₃-C₇ cycloalkyl, aryl, heteroaryl, OR⁶ and NR⁶R⁷, S(O)ₙR⁵ (where n=0, 1 or 2), CONR⁶R⁷, NR⁶COR⁷, SO₂NR⁶R⁷ and NR⁶SO₂R⁷; or

R⁹ and R¹⁰ together with the nitrogen atom to which they are attached can form a 3-8 membered saturated heterocylic ring optionally containing one or more atoms selected from O, S(O)ₙ (where n=0, 1 or 2), NR¹¹, and itself optionally substituted by halogen or C₁-C₃ alkyl; and

R¹¹ represents a hydrogen atom, C₁₋₆alkyl, C₃-C₇ cycloalkyl, SO₂R⁵ or COC₁-C₄ alkyl, provided that:
When Y is O and A=phenyl, then B is not aryl or a six membered heterocyclic aromatic ring containing one or more nitrogen atoms or a 6,6 or 6,5 fused bicycle containing one or more O, N, S atoms, and
When Y is O and B is phenyl or a 6,6 or 6,5 fused bicycle containing one or more O, N, S atoms, then A is not aryl.

Examples of aryl include phenyl and naphthyl.

Heteroaryl is defined as a 5-7 member aromatic ring or can be 6,6- or 6,5-fused bicyclic ring optionally containing one or more heteroatoms selected from N, S, O. The bicyclic ring may be linked through carbon or nitrogen and may be attached through the 5 or 6 membered ring and can be fully or partially saturated.

Examples include pyridine, pyrimidine, thiazole, oxazole, pyrazole, imidazole, furan, isoxazole, pyrrole, isothiazole and azulene, naphthyl, indene, quinoline, isoquinoline, indole, indolizine, benzo[b]furan, benzo[b]thiophene, 1H-indazole, benzimidazole, benzthiazole, benzoxazole, purine, 4H-quinolizine, cinnoline, phthalazine, quinazoline, quinoxaline, 1,8-naphthyridine, pteridine and quinolone.

Aryl or heteroaryl groups can be optionally substituted by one or more substituents independently selected from hydrogen, halogen, CN, OH, SH, nitro, CO₂R⁶, SO₂R⁸, OR⁸, SR⁸, SOR⁸, SO₂NR⁹R¹⁰, CONR⁹R¹⁰, NR⁹R¹⁰, NHSO₂R⁸, NR⁸SO₂R⁸, NR⁸CO₂R⁸, NHCOR⁸, NR⁸COR⁸, NR⁶CONR⁶R⁷, NR⁶SO₂NR⁶R⁷, aryl, heteroaryl, C₂-C₆ alkenyl, C₂-C₆ alkynyl, C₃-C₇ cycloalkyl or C₁-₆alkyl, the latter four groups being optionally substituted by one or more substituents independently selected from halogen, C₃-C₇ cycloalkyl, OR⁶, NR⁶R⁷, S(O)ₙR⁵ (where n is 0, 1 or 2), CONR⁶R⁷, NR⁶COR⁷, SO₂NR⁶R⁷ and NR⁶SO₂R⁵.

In the context of the present specification, unless otherwise indicated, an alkyl or alkenyl group or an alkyl or alkenyl moiety in a substituent group may be linear or branched.

Heterocyclic rings as defined for R¹, R² and R⁹ and R¹⁰ means saturated heterocycles, examples include morpholine, azetidine, pyrrolidine, piperidine and piperazine.

In a preferred embodiment X is YCR¹R². Preferably X is CH₂CH₂, CH₂S, CH₂NH, CH₂NMe, CH₂O, CH₂, CH═CH or CHCH₃O. In one embodiment of the invention X is CH₂CH₂, CH₂S, CH₂NH, CH₂NMe, CH₂, CH═CH.

Preferably A is phenyl or a six membered heterocyclic aromatic ring containing one or more nitrogen atoms, in particular pyridyl, substituted in the para position to the acid with halogen, trifluoromethyl, cyano, amino or C₁₋₃ alkyl.

Preferably B is isoxazolyl, phenyl, thienyl, furyl, pyrazolyl or indolyl, each optionally substituted as defined above.

Preferred substituents for A and B groups are those of the examples herein.

Preferably R¹ and R² are independently hydrogen or C₁₋₃ alkyl.

Preferred compounds of the invention include:
(4-Chloro-2-isoxazol-5-ylphenoxy)acetic acid
N-(5-Chlorobiphenyl-2-yl)glycine
3-(5-Chlorobiphenyl-2-yl)propanoic acid
(2E)-3-(5-Chlorobiphenyl-2-yl)acrylic acid
N-(5-Chlorobiphenyl-2-yl)-N-methylglycine
(5-Chlorobiphenyl-2-yl)acetic acid
{[5-Chloro-4′-(ethylthio)biphenyl-2-yl]thio}acetic acid
[5-Chloro-4′-(ethylsulfonyl)-2′-methylbiphenyl-2-yl]acetic acid
N-[4′-(Ethylsulfonyl)-5-(trifluoromethyl)biphenyl-2-yl]glycine
3-[4′-(Ethylsulfonyl)-2′-methyl-5-(trifluoromethyl)biphenyl-2-yl]propanoic acid
({2-[4-(Ethylsulfonyl)-2-methylphenyl]-6-methylpyridin-3-yl}oxy)acetic acid
[2-(2-Cyano-3-thienyl)-4-(trifluoromethyl)phenoxy]acetic acid
[2-(2-Furyl)-4-(trifluoromethyl)phenoxy]acetic acid
[2-(2-Chloro-3-thienyl)-4-(trifluoromethyl)phenoxy]acetic acid
[2-(2,5-Dichloro-3-thienyl)-4-(trifluoromethyl)phenoxy]acetic acid
[2-(2-Thienyl)-4-(trifluoromethyl)phenoxy]acetic acid
[2-(3-Thienyl)-4-(trifluoromethyl)phenoxy]acetic acid
[2-(5-Acetyl-2-thienyl)-4-(trifluoromethyl)phenoxy]acetic acid
[(5-Chloro-3′-cyanobiphenyl-2-yl)thio]acetic acid
(2S)-2-[2-[1-Methyl-3-(trifluoromethyl)-1H-pyrazol-5-yl]-4-(trifluoromethyl)phenoxy] propanoic acid
(2S)-2-[4-(Trifluoromethyl)-2-(1,3,5-trimethyl-1H-pyrazol-4-yl)phenoxy]propanoic acid
(2S)-2-[2-[1-Methyl-5-(trifluoromethyl)-1H-pyrazol-3-yl]-4-(trifluoromethyl)phenoxy] propanoic acid
(2S)-2-[2-{1-[(Dimethylamino)sulfonyl]-3-methyl-1H-pyrazol-4-yl} -4-(trifluoromethyl) phenoxy]propanoic acid
N-[4-Chloro-2-(5-methoxy-1H-indol-1-yl)phenyl]glycine
N-[4-Chloro-2-(5-cyano-1H-indol-1-yl)phenyl]glycine
({2-[2-Chloro-4-(methylsulfonyl)phenyl]-6-methylpyridin-3-yl}oxy)acetic acid
{[2-(3-Cyanophenyl)pyridin-3-yl]oxy}acetic acid
(2S)-2-({2-[2-Chloro-4-(methylsulfonyl)phenyl]-6-methylpyridin-3-yl}oxy)propanoic acid
{[6-Amino-2-(3-cyanophenyl)pyridin-3-yl]oxy}acetic acid
N-{4-Chloro-2-[5-(methylsulfonyl)-1H-indol-1-yl]phenyl}glycine,
3-[4′-(Methylsulfonyl)-3′,5-bis(trifluoromethyl)biphenyl-2-yl]propanoic acid
3-(5-Chloro-3′-cyanobiphenyl-2-yl)propanoic acid
3-[2′,5-Dichloro-4′-(methylsulfonyl)biphenyl-2-yl]propanoic acid
3-[3′-Fluoro-4′-(pyrrolidin-1-ylcarbonyl)-5-(trifluoromethyl)biphenyl-2-yl]propanoic acid
3-[2′-Chloro-4′-(methylsulfonyl)-5-(trifluoromethyl)biphenyl-2-yl]propanoic acid
3-[4′-(Ethylsulfonyl)-3′,5-bis(trifluoromethyl)biphenyl-2-yl]propanoic acid
3-[3′-Cyano-5′-fluoro-5-(trifluoromethyl)biphenyl-2-yl]propanoic acid
3-[3′-Cyano-5-(trifluoromethyl)biphenyl-2-yl]propanoic acid
3-[5-Chloro-3′-fluoro-4′-(phenylsulfonyl)biphenyl-2-yl]propanoic acid
3-[5-Chloro-4′-(pyridin-2-ylsulfonyl)biphenyl-2-yl]propanoic acid
and pharmaceutically acceptable salts thereof.

Certain compounds of formula (I) are capable of existing in stereoisomeric forms. It will be understood that the invention encompasses all geometric and optical isomers of the compounds of formula (I) and mixtures thereof including racemates. Tautomers and mixtures thereof also form an aspect of the present invention.

The compound of formula (I) above may be converted to a pharmaceutically acceptable salt or solvate thereof, preferably a basic addition salt such as sodium, potassium, calcium, aluminium, lithium, magnesium, zinc, benzathine, chloroprocaine, choline, iethanolamine, ethanolamine, ethyldiamine, meglumine, tromethamine or procaine, or an acid addition salt such as a hydrochloride, hydrobromide, phosphate, acetate, fumarate, maleate, tartrate, citrate, oxalate, methanesulphonate or p-toluenesulphonate.

It will be appreciated by those skilled in the art that in the processes of the present invention certain functional groups in the starting reagents or intermediate compound may need to be protected by protecting groups. Thus, the preparation of the compound of formula (I) may involve, at an appropriate stage, the removal of one or more protecting groups. The protection and deprotection of functional groups is fully described in ‘Protective Groups in Organic Chemistry’, edited by J. W. F. McOmie, Plenum Press (1973), and ‘Protective Groups in Organic Synthesis’, 3rd edition, T. W. Greene & P. G. M. Wuts, Wiley-Interscience (1999).

Compounds of formula (I) can be prepared by reaction of a compound of formula (II): 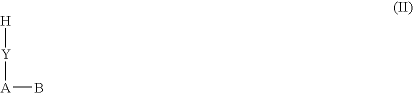
in which Y=O, S or NR³ and A and B are as defined in formula (I) or are protected derivatives thereof, with a compound of formula (III):
L-CR¹R²CO₂R¹² (III)

Where R¹ and R² are as defined in formula (I) or are protected derivatives thereof, R¹² is H or C₁-C₁₀ alkyl group and L is a leaving group, and optionally thereafter in any order:
removing any protecting group
hydrolysing the ester group R¹² to the corresponding acid
oxidation of sulphides to sulphoxides or sulphones
forming a pharmaceutically acceptable salt.

The reaction can be carried out in a suitable solvent such as DMF using a base such as potassium carbonate or the like. Suitable groups R¹² include C₁₋₆ alkyl groups such as methyl, ethyl or tert-butyl. Suitable L is a leaving group such as halo, in particular chlorine or bromine. L may also be hydroxy so that a Mitsunobu reaction may be performed with compound (II) using for example triphenylphosphine and diethyl azodicarboxylate. Hydrolysis of the ester group R¹² can be carried out using routine procedures, for example treatment of methyl and ethyl esters with aqueous sodium hydroxide, and treatment of tert-butyl esters with acids such as trifluoroacetic acid.

Compounds of formula (I) can be prepared by a Suzuki reaction of a compound of formula (IV) with a compound of formula (V) followed by removal of any protecting groups: 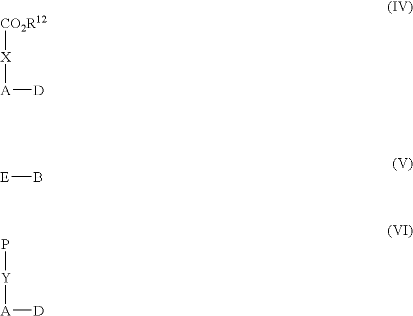
in which A, B and X are as defined in formula (I) or are protected derivatives thereof and D is a boronic acid or ester when E is a halogen, mesylate or triflate or alternatively D is a halogen, mesylate or triflate when E is a boronic acid or ester.

The reaction can be carried out in a suitable solvent such as dioxane using a palladium catalyst such as [1,1-bis(diphenylphosphino)ferrocene]dichloropalladium and a base such as cesium fluoride, preferably at elevated temperatures.

Similarly a compound of formula (II) can be prepared via a suzuki reaction using a compound of formula (VI), where P=hydrogen or a protecting group.

A compound of formula (VII) may be prepared by method A or B 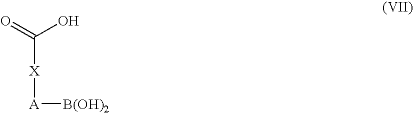
Method A

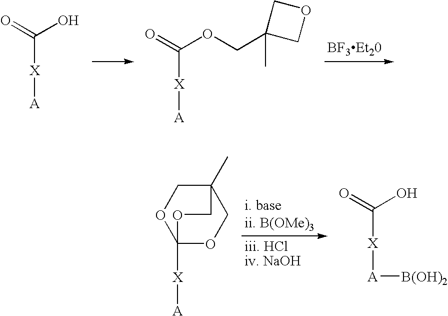

The acid was first converted to the acid chloride, using for example oxalylchloride in DCM at RT, then reacted with 3-methyl-3-oxetanemethanol in the presence of a base such as triethylamine to give the ester. The oxetane ester is then rearranged to the OBO ester using boron trifluoride diethyletherate in DCM at −78° C. to RT. Deprotonation with a base such as sec -butyl lithium at low temperature followed by quenching with trimethylborate gave the protected diacid which was then deprotected using HCl then sodium hydroxide
Method B

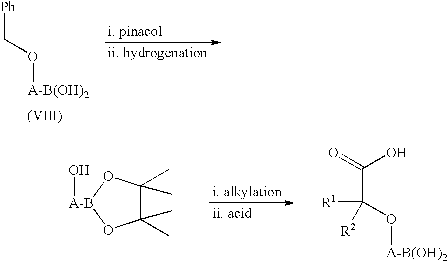

A compound of formula (VIII) and pinacol can be stirred at RT in a suitable solvent such as diethylether to give the boronate ester. The benzyl group may be removed by hydrogenation at RT using palladium on carbon as catalyst then alkylated with a compound of formula (III) using a base or mitsunobu conditions. Treatment with acid such as HCl or trifluoroacetic acid then removes the protecting groups.

Compounds of formula (I), where Y=bond, may be prepared using the general route A: 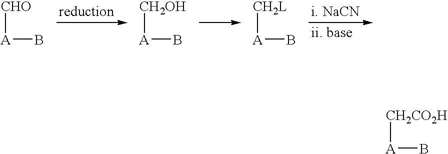
Route A

in which L is a leaving group. The formyl group can then be reduced to the alcohol using a suitable reducing agent such as sodium borohydride in ethanol. The alcohol can be converted into a leaving group such as a mesylate, using methanesulphonyl chloride and triethylamine and then displaced with cyanide. The nitrile can be hydrolysed to the acid under basic conditions , for example potassium hydroxide, at elevated temperatures

Compounds of formula (I), where Y=CR¹R² and X=CR³=CR⁴, may be prepared using the general route B: 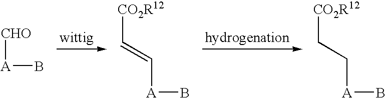
Route B

Alternatively compounds of formula (I) where Y=CR¹R² may be prepared using the general route B (i): 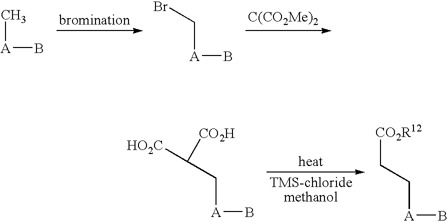
Route B (i)

In a further aspect, the present invention provides the use of a compound of formula (I), pharmaceutically acceptable salt or solvate thereof for use in therapy.

The compounds of formula (I) have activity as pharmaceuticals, in particular as modulators of CRTh2 receptor activity, and may be used in the treatment (therapeutic or prophylactic) of conditions/diseases in human and non-human animals which are exacerbated or caused by excessive or unregulated production of PGD₂ and its metabolites. Examples of such conditions/diseases include:
1. respiratory tract: obstructive diseases of the airways including: asthma, including bronchial, allergic, intrinsic, extrinsic, exercise-induced, drug-induced (including aspirin and NSAID-induced) and dust-induced asthma, both intermittent and persistent and of all severities, and other causes of airway hyper-responsiveness; chronic obstructive pulmonary disease (COPD); bronchitis, including infectious and eosinophilic bronchitis; emphysema; bronchiectasis; cystic fibrosis; sarcoidosis; farmer's lung and related diseases; hypersensitivity pneumonitis; lung fibrosis, including cryptogenic fibrosing alveolitis, idiopathic interstitial pneumonias, fibrosis complicating anti-neoplastic therapy and chronic infection, including tuberculosis and aspergillosis and other fungal infections; complications of lung transplantation; vasculitic and thrombotic disorders of the lung vasculature, and pulmonary hypertension; antitussive activity including treatment of chronic cough associated with inflammatory and secretory conditions of the airways, and iatrogenic cough; acute and chronic rhinitis including rhinitis medicamentosa, and vasomotor rhinitis; perennial and seasonal allergic rhinitis including rhinitis nervosa (hay fever); nasal polyposis; acute viral infection including the common cold, and infection due to respiratory syncytial virus, influenza, coronavirus (including SARS) and adenovirus;
2. bone and joints: arthritides associated with or including o steoarthritis/osteoarthrosis, both primary and secondary to, for example, congenital hip dysplasia; cervical and lumbar spondylitis, and low back and neck pain; rheumatoid arthritis and Still's disease; seronegative spondyloarthropathies including ankylosing spondylitis, psoriatic arthritis, reactive arthritis and undifferentiated spondarthropathy; septic arthritis and other infection-related arthopathies and bone disorders such as tuberculosis, including Potts' disease and Poncet's syndrome; acute and chronic crystal-induced synovitis including urate gout, calcium pyrophosphate deposition disease, and calcium apatite related tendon, bursal and synovial inflammation; Behcet's disease; primary and secondary Sjogren's syndrome; systemic sclerosis and limited scleroderna; systemic lupus erythematosus, mixed connective tissue disease, and undifferentiated connective tissue disease; inflammatory myopathies including dermatomyositits and polymyositis; polymalgia rheumatica; juvenile arthritis including idiopathic inflammatory arthritides of whatever joint distribution and associated syndromes, and rheumatic fever and its systemic complications; vasculitides including giant cell arteritis, Takayasu's arteritis, Churg-Strauss syndrome, polyarteritis nodosa, microscopic polyarteritis, and vasculitides associated with viral infection, hypersensitivity reactions, cryoglobulins, and paraproteins; low back pain; Familial Mediterranean fever, Muckle-Wells syndrome, and Familial Hibernian Fever, Kikuchi disease; drug-induced arthalgias, tendonititides, and myopathies;
3. pain and connective tissue remodelling of musculoskeletal disorders due to injury [for example sports injury] or disease: arthitides (for example rheumatoid arthritis, osteoarthritis, gout or crystal arthropathy), other joint disease (such as intervertebral disc degeneration or temporomandibular joint degeneration), bone remodelling disease (such as osteoporosis, Paget's disease or osteonecrosis), polychondritits, scleroderma, mixed connective tissue disorder, spondyloarthropathies or periodontal disease (such as periodontitis);
4. skin: psoriasis, atopic dermatitis, contact dermatitis or other eczematous dermatoses, and delayed-type hypersensitivity reactions; phyto- and photodermatitis; seborrhoeic dermatitis, dermatitis herpetiformis, lichen planus, lichen sclerosus et atrophica, pyoderma gangrenosum, skin sarcoid, discoid lupus erythematosus, pemphigus, pemphigoid, epidermolysis bullosa, urticaria, angioedema, vasculitides, toxic erythemas, cutaneous eosinophilias, alopecia areata, male-pattern baldness, Sweet's syndrome, Weber-Christian syndrome, erythema multiforme; cellulitis, both infective and non-infective; panniculitis; cutaneous lymphomas, non-melanoma skin cancer and other dysplastic lesions; drug-induced disorders including fixed drug eruptions;
5. eyes: blepharitis; conjunctivitis, including perennial and vernal allergic conjunctivitis; iritis; anterior and posterior uveitis; choroiditis; autoimmune; degenerative or inflammatory disorders affecting the retina; ophthalmitis including sympathetic ophthalmitis; sarcoidosis; infections including viral, fungal, and bacterial;
6. gastrointestinal tract: glossitis, gingivitis, periodontitis; oesophagitis, including reflux; eosinophilic gastro-enteritis, mastocytosis, Crohn's disease, colitis including ulcerative colitis, proctitis, pruritis ani; coeliac disease, irritable bowel syndrome, and food-related allergies which may have effects remote from the gut (for example migraine, rhinitis or eczema);
7. abdominal: hepatitis, including autoimmune, alcoholic and viral; fibrosis and cirrhosis of the liver; cholecystitis; pancreatitis, both acute and chronic; 8. genitourinary: nephritis including interstitial and glomerulonephritis; nephrotic syndrome; cystitis including acute and chronic (interstitial) cystitis and Hunner's ulcer; acute and chronic urethritis, prostatitis, epididymitis, oophoritis and salpingitis; vulvo-vaginitis; Peyronie's disease; erectile dysfunction (both male and female);
9. allograft rejection: acute and chronic following, for example, transplantation of kidney, heart, liver, lung, bone marrow, skin or cornea or following blood transfusion; or chronic graft versus host disease;
10. CNS: Alzheimer's disease and other dementing disorders including CJD and nvCJD; amyloidosis; multiple sclerosis and other demyelinating syndromes; cerebral atherosclerosis and vasculitis; temporal arteritis; myasthenia gravis; acute and chronic pain (acute, intermittent or persistent, whether of central or peripheral origin) including visceral pain, headache, migraine, trigeminal neuralgia, atypical facial pain, joint and bone pain, pain arising from cancer and tumor invasion, neuropathic pain syndromes including diabetic, post-herpetic, and HIV-associated neuropathies; neurosarcoidosis; central and peripheral nervous system complications of malignant, infectious or autoimmune processes;
11. other auto-immune and allergic disorders including Hashimoto's thyroiditis, Graves' disease, Addison's disease, diabetes mellitus, idiopathic thrombocytopaenic purpura, eosinophilic fasciitis, hyper-IgE syndrome, antiphospholipid syndrome;
12. other disorders with an inflammatory or immunological component; including acquired immune deficiency syndrome (AIDS), leprosy, Sezary syndrome, and paraneoplastic syndromes;
13. cardiovascular: atherosclerosis, affecting the coronary and peripheral circulation; pericarditis; myocarditis, inflammatory and auto-immune cardiomyopathies including myocardial sarcoid; ischaemic reperfusion injuries; endocarditis, valvulitis, and aortitis including infective (for example syphilitic); vasculitides; disorders of the proximal and peripheral veins including phlebitis and thrombosis, including deep vein thrombosis and complications of varicose veins;
14. oncology: treatment of common cancers including prostate, breast, lung, ovarian, pancreatic, bowel and colon, stomach, skin and brain tumors and malignancies affecting the bone marrow (including the leukaemias) and lymphoproliferative systems, such as Hodgkin's and non-Hodgkin's lymphoma; including the prevention and treatment of metastatic disease and tumour recurrences, and paraneoplastic syndromes; and,
15. gastrointestinal tract: Coeliac disease, proctitis, eosinopilic gastro-enteritis, mastocytosis, Crohn's disease, ulcerative colitis, microscopic colitis, indeterminant colitis, irritable bowel disorder, irritable bowel syndrome, non-inflammatory diarrhea, food-related allergies which have effects remote from the gut, e.g., migraine, rhinitis and eczema.
16. Diseases associated with raised levels of PGD₂ or its metabolites.

Thus, the present invention provides a compound of formula (I), or a pharmaceutically-acceptable salt or solvate thereof, as hereinbefore defined for use in therapy.

Preferably the compounds of the invention are used to treat diseases in which the chemokine receptor belongs to the CRTh2 receptor subfamily.

Particular conditions which can be treated with the compounds of the invention are asthma, rhinitis and other diseases in which raised levels of PGD₂ or its metabolites. It is preferred that the compounds of the invention are used to treat asthma.

In a further aspect, the present invention provides the use of a compound of formula (I), or a pharmaceutically acceptable salt or solvate thereof, as hereinbefore defined in the manufacture of a medicament for use in therapy.

The invention further relates to combination therapies wherein a compound of the invention, or a pharmaceutically acceptable salt thereof, or a pharmaceutical composition or formulation comprising a compound of the invention, is administered concurrently or sequentially or as a combined preparation with another therapeutic agent or agents, for the treatment of one or more of the conditions listed.

In particular, for the treatment of the inflammatory diseases such as (but not restricted to) rheumatoid arthritis, osteoarthritis, asthma, allergic rhinitis, chronic obstructive pulmonary disease (COPD), psoriasis, and inflammatory bowel disease, the compounds of the invention may be combined with agents listed below.

Non-steroidal anti-inflammatory agents (hereinafter NSAIDs) including non-selective cyclo-oxygenase COX-1/COX-2 inhibitors whether applied topically or systemically (such as piroxicam, diclofenac, propionic acids such as naproxen, flurbiprofen, fenoprofen, ketoprofen and ibuprofen, fenamates such as mefenamic acid, indomethacin, sulindac, azapropazone, pyrazolones such as phenylbutazone, salicylates such as aspirin); selective COX-2 inhibitors (such as meloxicam, celecoxib, rofecoxib, valdecoxib, lumarocoxib, parecoxib and etoricoxib); cyclo-oxygenase inhibiting nitric oxide donors (CINODs); glucocorticosteroids (whether administered by topical, oral, intramuscular, intravenous, or intra-articular routes); methotrexate; leflunomide; hydroxychloroquine; d-penicillamine; auranofin or other parenteral or oral gold preparations; analgesics; diacerein; intra-articular therapies such as hyaluronic acid derivatives; and nutritional supplements such as glucosamine.

The present invention still further relates to the combination of a compound of the invention, or a pharmaceutically acceptable salt thereof, together with a cytokine or agonist or antagonist of cytokine function, (including agents which act on cytokine signalling pathways such as modulators of the SOCS system) including alpha-, beta-, and gamma-interferons; insulin-like growth factor type I (IGF-1); interleukins (IL) including IL1 to 17, and interleukin antagonists or inhibitors such as anakinra; tumour necrosis factor alpha (TNF-α) inhibitors such as anti-TNF monoclonal antibodies (for example infliximab; adalimumab, and CDP-870) and TNF receptor antagonists including immunoglobulin molecules (such as etanercept) and low-molecular-weight agents such as pentoxyfylline.

In addition the invention relates to a combination of a compound of the invention, or a pharmaceutically acceptable salt thereof, with a monoclonal antibody targeting B-Lymphocytes (such as CD20 (rituximab), MRA-aIL16R and T-Lymphocytes, CTLA4-Ig, HuMax I1-15).

The present invention still further relates to the combination of a compound of the invention, or a pharmaceutically acceptable salt thereof, with a modulator of chemokine receptor function such as an antagonist of CCR1, CCR2, CCR2A, CCR2B, CCR3, CCR4, CCR5, CCR6, CCR7, CCR8, CCR9, CCR10 and CCR11 (for the C-C family); CXCR1, CXCR2, CXCR3, CXCR4 and CXCR5 (for the C-X-C family) and CX₃CR1 for the C-X₃-C family.

The present invention further relates to the combination of a compound of the invention, or a pharmaceutically acceptable salt thereof, with an inhibitor of matrix metalloprotease (MMPs), i.e., the stromelysins, the collagenases, and the gelatinases, as well as aggrecanase; especially collagenase-1 (MMP-1), collagenase-2 (MMP-8), collagenase-3 (MMP-13), stromelysin-1 (MMP-3), stromelysin-2 (MMP-10), and stromelysin-3 (MMP-11) and MMP-9 and MMP-12, including agents such as doxycycline.

The present invention still further relates to the combination of a compound of the invention, or a pharmaceutically acceptable salt thereof, and a leukotriene biosynthesis inhibitor, 5-lipoxygenase (5-LO) inhibitor or 5-lipoxygenase activating protein (FLAP) antagonist such as; zileuton; ABT-761; fenleuton; tepoxalin; Abbott-79175; Abbott-85761; a N-(5-substituted)-thiophene-2-alkylsulfonamide; 2,6-di-tert-butylphenolhydrazones; a methoxytetrahydropyrans such as Zeneca ZD-2138; the compound SB-210661; a pyridinyl-substituted 2-cyanonaphthalene compound such as L-739,010; a 2-cyanoquinoline compound such as L-746,530; or an indole or quinoline compound such as MK-591, MK-886, and BAY x 1005.

The present invention further relates to the combination of a compound of the invention, or a pharmaceutically acceptable salt thereof, and a receptor antagonist for leukotrienes (LT) B4, LTC4, LTD4, and LTE4. selected from the group consisting of the phenothiazin-3-1s such as L-651,392; amidino compounds such as CGS-25019c; benzoxalamines such as ontazolast; benzenecarboximidamides such as BIIL 284/260; and compounds such as zafirlukast, ablukast, montelukast, pranlukast, verlukast (MK-679), RG-12525, Ro-245913, iralukast (CGP 45715A), and BAY x 7195.

The present invention still further relates to the combination of a compound of the invention, or a pharmaceutically acceptable salt thereof, and a phosphodiesterase (PDE) inhibitor such as a methylxanthanine including theophylline and aminophylline; a selective PDE isoenzyme inhibitor including a PDE4 inhibitor an inhibitor of the isoform PDE4D, or an inhibitor of PDE5.

The present invention further relates to the combination of a compound of the invention, or a pharmaceutically acceptable salt thereof, and a histamine type 1 receptor antagonist such as cetirizine, loratadine, desloratadine, fexofenadine, acrivastine, terfenadine, astemizole, azelastine, levocabastine, chlorpheniramine, promethazine, cyclizine, or mizolastine; applied orally, topically or parenterally.

The present invention still further relates to the combination of a compound of the invention, or a pharmaceutically acceptable salt thereof, and a proton pump inhibitor (such as omeprazole) or a gastroprotective histamine type 2 receptor antagonist.

The present invention further relates to the combination of a compound of the invention, or a pharmaceutically acceptable salt thereof, and an antagonist of the histamine type 4 receptor.

The present invention still further relates to the combination of a compound of the invention, or a pharmaceutically acceptable salt thereof, and an alpha-1/alpha-2 adrenoceptor agonist vasoconstrictor sympathomimetic agent, such as propylhexedrine, phenylephrine, phenylpropanolamine, ephedrine, pseudoephedrine, naphazoline hydrochloride, oxymetazoline hydrochloride, tetrahydrozoline hydrochloride, xylometazoline hydrochloride, tramazoline hydrochloride or ethylnorepinephrine hydrochloride.

The present invention further relates to the combination of a compound of the invention, or a pharmaceutically acceptable salt thereof, and an anticholinergic agents including muscarinic receptor (M1, M2, and M3) antagonist such as atropine, hyoscine, glycopyrrrolate, ipratropium bromide, tiotropium bromide, oxitropium bromide, pirenzepine or telenzepine.

The present invention still further relates to the combination of a compound of the invention, or a pharmaceutically acceptable salt thereof, and a beta-adrenoceptor agonist (including beta receptor subtypes 1-4) such as isoprenaline, salbutamol, formoterol, salmeterol, terbutaline, orciprenaline, bitolterol mesylate, or pirbuterol, or a chiral enantiomer thereof.

The present invention further relates to the combination of a compound of the invention, or a pharmaceutically acceptable salt thereof, and a chromone, such as sodium cromoglycate or nedocromil sodium.

The present invention still further relates to the combination of a compound of the invention, or a pharmaceutically acceptable salt thereof, with a glucocorticoid, such as flunisolide, triamcinolone acetonide, beclomethasone dipropionate, budesonide, fluticasone propionate, ciclesonide or mometasone furoate.

The present invention further relates to the combination of a compound of the invention, or a pharmaceutically acceptable salt thereof, with an agent that modulates a nuclear hormone receptor such as PPARs.

The present invention still further relates to the combination of a compound of the invention, or a pharmaceutically acceptable salt thereof, together with an immunoglobulin (Ig) or Ig preparation or an antagonist or antibody modulating Ig function such as anti-IgE (for example omalizumab).

The present invention further relates to the combination of a compound of the invention, or a pharmaceutically acceptable salt thereof, and another systemic or topically-applied anti-inflammatory agent, such as thalidomide or a derivative thereof, a retinoid, dithranol or calcipotriol.

The present invention still further relates to the combination of a compound of the invention, or a pharmaceutically acceptable salt thereof, and combinations of aminosalicylates and sulfapyridine such as sulfasalazine, mesalazine, balsalazide, and olsalazine; and immunomodulatory agents such as the thiopurines, and corticosteroids such as budesonide.

The present invention further relates to the combination of a compound of the invention, or a pharmaceutically acceptable salt thereof, together with an antibacterial agent such as a penicillin derivative, a tetracycline, a macrolide, a beta-lactam, a fluoroquinolone, etronidazole, an inhaled aminoglycoside; an antiviral agent including acyclovir, famciclovir, valaciclovir, ganciclovir, cidofovir, amantadine, rimantadine, ribavirin, zanamavir and oseltarnavir; a protease inhibitor such as indinavir, nelfinavir, ritonavir, and saquinavir; a nucleoside reverse transcriptase inhibitor such as didanosine, lamivudine, stavudine, zalcitabine or zidovudine; or a non-nucleoside reverse transcriptase inhibitor such as nevirapine or efavirenz.

The present invention still further relates to the combination of a compound of the invention, or a pharmaceutically acceptable salt thereof, and a cardiovascular agent such as a calcium channel blocker, a beta-adrenoceptor blocker, an angiotensin-converting enzyme (ACE) inhibitor, an angiotensin-2 receptor antagonist; a lipid lowering agent such as a statin or a fibrate; a modulator of blood cell morphology such as pentoxyfylline; thrombolytic, or an anticoagulant such as a platelet aggregation inhibitor.

The present invention further relates to the combination of a compound of the invention, or a pharmaceutically acceptable salt thereof, and a CNS agent such as an antidepressant (such as sertraline), an anti-Parkinsonian drug (such as deprenyl, L-dopa, ropinirole, pramipexole, a MAOB inhibitor such as selegine and rasagiline, a comP inhibitor such as tasmar, an A-2 inhibitor, a dopamine reuptake inhibitor, an NMDA antagonist, a nicotine agonist, a dopamine agonist or an inhibitor of neuronal nitric oxide synthase), or an anti-Alzheimer's drug such as donepezil, rivastigmine, tacrine, a COX-2 inhibitor, propentofylline or metrifonate.

The present invention still further relates to the combination of a compound of the invention, or a pharmaceutically acceptable salt thereof, and an agent for the treatment of acute or chronic pain, such as a centrally or peripherally-acting analgesic (for example an opioid or derivative thereof), carbamazepine, phenytoin, sodium valproate, amitryptiline or other anti-depressant agent-s, paracetamol, or a non-steroidal anti-inflammatory agent.

The present invention further relates to the combination of a compound of the invention, or a pharmaceutically acceptable salt thereof, together with a parenterally or topically-applied (including inhaled) local anaesthetic agent such as lignocaine or a derivative thereof.

A compound of the present invention, or a pharmaceutically acceptable salt thereof, can also be used in combination with an anti-osteoporosis agent including a hormonal agent such as raloxifene, or a biphosphonate such as alendronate.

The present invention still further relates to the combination of a compound of the invention, or a phannaceutically acceptable salt thereof, together with a: (i) tryptase inhibitor; (ii) platelet activating factor (PAF) antagonist; (iii) interleukin converting enzyme (ICE) inhibitor; (iv) IMPDH inhibitor; (v) adhesion molecule inhibitors including VLA-4 antagonist; (vi) cathepsin; (vii) kinase inhibitor such as an inhibitor of tyrosine kinase (such as Btk, Itk, Jak3 or MAP, for example Gefitinib or Imatinib mesylate), a serine / threonine kinase (such as an inhibitor of a MAP kinase such as p38, JNK, protein kinase A, B or C, or IKK), or a kinase involved in cell cycle regulation (such as a cylin dependent kinase); (viii) glucose-6 phosphate dehydrogenase inhibitor; (ix) kinin-B.sub1.- or B.sub2.-receptor antagonist; (x) anti-gout agent, for example colchicine; (xi) xanthine oxidase inhibitor, for example allopurinol; (xii) uricosuric agent, for example probenecid, sulfinpyrazone or benzbromarone; (xiii) growth hormone secretagogue; (xiv) transforming growth factor (TGFβ); (xv) platelet-derived growth factor (PDGF); (xvi) fibroblast growth factor for example basic fibroblast growth factor (bFGF); (xvii) granulocyte macrophage colony stimulating factor (GM-CSF); (xviii) capsaicin cream; (xix) tachykinin NK.sub1. or NK.sub3. receptor antagonist such as NKP-608C, SB-233412 (talnetant) or D-4418; (xx) elastase inhibitor such as UT-77 or ZD-0892; (xxi) TNF-alpha converting enzyme inhibitor (TACE); (xxii) induced nitric oxide synthase (iNOS) inhibitor; (xxiii) chemoattractant receptor-homologous molecule expressed on TH2 cells, (such as a CRTH2 antagonist); (xxiv) inhibitor of P38; (xxv) agent modulating the flnction of Toll-like receptors (TLR), (xxvi) agent modulating the activity of purinergic receptors such as P2X7; or (xxvii) inhibitor of transcription factor activation such as NFkB, API, or STATS.

A compound of the invention, or a pharmaceutically acceptable salt thereof, can also be used in combination with an existing therapeutic agent for the treatment of cancer, for example suitable agents include:
(i) an antiproliferative/antineoplastic drug or a combination thereof, as used in medical oncology, such as an alkylating agent (for example cis-platin, carboplatin, cyclophosphamide, nitrogen mustard, melphalan, chlorambucil, busulphan or a nitrosourea); an antimetabolite (for example an antifolate such as a fluoropyrimidine like 5-fluorouracil or tegafur, raltitrexed, methotrexate, cytosine arabinoside, hydroxyurea, gemcitabine or paclitaxel); an antitumour antibiotic (for example an anthracycline such as adriamycin, bleomycin, doxorubicin, daunomycin, epirubicin, idarubicin, mitomycin-C, dactinomycin or mithramycin); an antimitotic agent (for example a vinca alkaloid such as vincristine, vinblastine, vindesine or vinorelbine, or a taxoid such as taxol or taxotere); or a topoisomerase inhibitor (for example an epipodophyllotoxin such as etoposide, teniposide, amsacrine, topotecan or a camptothecin);
(ii) a cytostatic agent such as an antioestrogen (for example tamoxifen, toremifene, raloxifene, droloxifene or iodoxyfene), an oestrogen receptor down regulator (for example fulvestrant), an antiandrogen (for example bicalutamide, flutamide, nilutamide or cyproterone acetate), a LHRH antagonist or LHRH agonist (for example goserelin, leuprorelin or buserelin), a progestogen (for example megestrol acetate), an aromatase inhibitor (for example as anastrozole, letrozole, vorazole or exeniestane) or an inhibitor of 5α-reductase such as finasteride;
(iii) an agent which inhibits cancer cell invasion (for example a metalloproteinase inhibitor like marimastat or an inhibitor of urokinase plasminogen activator receptor function);
(iv) an inhibitor of growth factor function, for example: a growth factor antibody (for example the anti-erbb2 antibody trastuzumab, or the anti-erbbl antibody cetuximab [C225]), a farnesyl transferase inhibitor, a tyrosine kinase inhibitor or a serine/threonine kinase inhibitor, an inhibitor of the epidermal growth factor family (for example an EGFR family tyrosine kinase inhibitor such as N-(3-chloro-4-fluorophenyl)-7-methoxy-6-(3-morpholinopropoxy)quinazolin-4-amine (gefitinib, AZD1839), N-(3-ethynylphenyl)-6,7-bis(2-methoxyethoxy)quinazolin-4-amine (erlotinib, OSI-774) or 6-acrylamido-N-(3-chloro-4-fluorophenyl)-7-(3-morpholinopropoxy)quinazolin-4-amine (Cl 1033)), an inhibitor of the platelet-derived growth factor family, or an inhibitor of the hepatocyte growth factor family;
(v) an antiangiogenic agent such as one which inhibits the effects of vascular endothelial growth factor (for example the anti-vascular endothelial cell growth factor antibody bevacizumab, a compound disclosed in WO 97/22596, WO 97/30035, WO 97/32856 or WO 98/13354), or a compound that works by another mechanism (for example linomide, an inhibitor of integrin αvβ3 function or an angiostatin);
(vi) a vascular damaging agent such as combretastation A4, or a compound disclosed in WO 99/02166, WO 00/40529, WO 00/41669, WO 01/92224, WO 02/04434 or WO 02/08213;
(vii) an agent used in antisense therapy, for example one directed to one of the targets listed above, such as ISIS 2503, an anti-ras antisense;
(viii) an agent used in a gene therapy approach, for example approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy; or
(ix) an agent used in an immunotherapeutic approach, for example ex-vivo and in-vivo approaches to increase the immunogenicity of patient tumour cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumour cell lines and approaches using anti-idiotypic antibodies.

In a still further aspect, the present invention provides the use of a compound of formula (I), or a pharmaceutically acceptable salt or solvate thereof, as hereinbefore defined in the manufacture of a medicament for the treatment of human diseases or conditions in which modulation of CRTh2 receptor activity is beneficial.

In the context of the present specification, the term “therapy” also includes “prophylaxis” unless there are specific indications to the contrary. The terms “therapeutic” and “therapeutically” should be construed accordingly.

The invention still further provides a method of treating diseases mediated by PGD2 or its metabolites wherein the prostanoid binds to its receptor (especially CRTh2) receptor, which comprises administering to a patient a therapeutically effective amount of a compound of formula (I), or a pharmaceutically acceptable salt, solvate or prodrug thereof, as hereinbefore defined.

The invention also provides a method of treating an inflammatory disease, especially psoriasis, in a patient suffering from, or at risk of, said disease, which comprises administering to the patient a therapeutically effective amount of a compound of formula (I), or a pharmaceutically acceptable salt or solvate thereof, as hereinbefore defined.

For the above-mentioned therapeutic uses the dosage administered will, of course, vary with the compound employed, the mode of administration, the treatment desired and the disorder indicated.

For the above-mentioned therapeutic uses the dosage administered will, of course, vary with the compound employed, the mode of administration, the treatment desired and the disorder indicated.

The compound of formula (I), prodrugs and pharmaceutically acceptable salts and solvates thereof may be used on their own but will generally be administered in the form of a pharmaceutical composition in which the formula (I) compound/salt/solvate (active ingredient) is in association with a pharmaceutically acceptable adjuvant, diluent or carrier. Depending on the mode of administration, the pharmaceutical composition will preferably comprise from 0.05 to 99% w (percent by weight), more preferably from 0.05 to 80% w, still more preferably from 0.10 to 70% w, and even more preferably from 0.10 to 50% w, of active ingredient, all percentages by weight being based on total composition.

The present invention also provides a pharmaceutical composition comprising a compound of formula (I), or a pharmaceutically acceptable salt or solvate thereof, as herein before defined, in association with a pharmaceutically acceptable adjuvant, diluent or carrier.

The pharmaceutical compositions may be administered topically (e.g. to the lung and/or airways or to the skin) in the form of solutions, suspensions, heptafluoroalkane aerosols and dry powder formulations; or systemically, e.g. by oral administration in the form of tablets, capsules, syrups, powders or granules, or by parenteral administration in the form of solutions or suspensions, or by subcutaneous administration or by rectal administration in the form of suppositories or transdermally. Preferably the compound of the invention is administered orally.

The invention will now be illustrated by the following non-limiting examples in which, unless stated otherwise:
(i) when given, ¹H NMR data is quoted in the form of delta values for major diagnostic protons, given in parts per million (ppm) relative to tetramethylsilane (TMS) as an internal standard;
(ii) mass spectra (MS): generally only ions which indicate the parent mass are reported, and unless otherwise stated the mass ion quoted is the positive mass ion—(M+H)⁺;
(iii) the title compounds of the examples and methods were named using the ACD/name (version 6.0) from Advanced Chemical Development Inc, Canada;
(iv) unless stated otherwise, reverse phase HPLC was conducted using a Symmetry, NovaPak or Ex-Terra reverse phase silica column;
(v) solvents were dried with MgSO₄ or Na₂SO₄

(vi) the following abbreviations are used:

| | |
|---|---|
| EtOAc | Ethyl acetate |
| DCM | Dichloromethane |
| NMP | N-methylpyrrolidine |
| DMF | N,N-dimethylformamide |
| THF | tetrahydrofuran |
| MCPBA | 3-chloroperoxybenzoic acid (Aldrich 77% max) |
| | Pd(dppf)Cl₂ [1,1′- |
| | Bis(diphenylphosphino)ferrocene]dichloropalladium(II), |
| | complex with dichloromethane |
| RT | room temperature |
| RPHPLC | reverse phase HPLC |
| wt | weight |

### EXAMPLE 1

### (4-Chloro-2-isoxazol-5-ylphenoxy)acetic acid

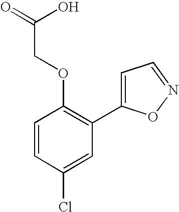

4-Chloro-2-(5-isoxazolyl)phenol (0.43 g), potassium carbonate (0.304 g) and tert-butyl bromoacetate (0.43 g) in DMF (8 ml) was vigorously stirred at RT for 18 h. The mixture was partitioned between ethyl acetate and water, the organic layer was washed with water, dried, and the solvent evaporated under reduced pressure. The residue was dissolved in a mixture of CM (5 ml) and trifluoroacetic acid (5 ml), left for 2 h then evaporated under reduced pressure. The residue was triturated with diethylether and filtered. Yield 0.29 g.

1H NMR DMSO-d6: δ 13.27 (1H, s), 8.73 (1H, s), 7.87 (1H, d), 7.55-7.52 (1H, m), 7.29 (1H, d), 7.21 (1H, d), 4.91 (2H, s).

MS: APCI (−ve) 252 (M−1)

### EXAMPLE 2

### N-(5-Chlorobiphenyl-2-yl)glycine

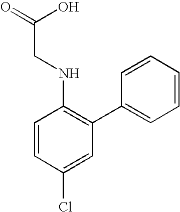

(i) 5-Chlorobiphenyl-2-amine

A mixture of 2-bromo-4-chloroaniline (4 g), sodium carbonate (6.1 g) phenylboronic acid (2.93 g) and tetrakis(triphenylphosphine)palladium(O) (0.5 g) in dioxane (30 ml) was heated under reflux for 48 h then cooled. The mixture was partitioned between ethyl acetate and water, the organic layer was washed with water, dried, and the solvent evaporated under reduced pressure. The residue was purified by chromatography on silica eluting with 5-10% ethyl acetate/isohexane, yield 0.99 g.

MS: APCI (+ve) 204/6

(ii) tert-Butyl N-(5-chlorobiphenyl-2-yl)glycinate

A mixture of the product from step (i) (0.94 g), sodium acetate (0.5 g) and tert-butylbromoacetate (0.6 ml) in ethanol (20 ml) was heated under reflux for 5 h. A further porton of sodium acetate (1 g) and tert-butylbromoacetate(1.2 ml) was added and the mixture heated under reflux overnight. The solvent was evaporated under reduced pressure and the residue partitioned between ethyl acetate and water, the organic layer was washed with water, dried, and the solvent evaporated under reduced pressure. The residue was purified by chromatography on silica eluting with 5% ethyl acetate/isohexane, yield 0.775 g.

MS: APCI (−ve) 260 (M-1-tBu)

(iii) N-(5-Chlorobiphenyl-2-yl)glycine

A solution of the product from step (ii) (0.77 g) in DCM (20 ml) and trifluoroacetic acid (10 ml) was stirred at RT for 4 h then evaporated under reduced pressure. The residue was partitioned between 2M sodium hydroxide solution and diethylether, the aqueous layer was acidified to pH4 and extracted with ethyl acetate. The ethyl acetate layer was washed with water, dried and evaporated under reduced pressure. The residue was purified by RPHPLC, yield 0.048 g.

1H NMR: DMSO-d6: δ 12.70 (1H, s), 7.52-7.39 (5H, m), 7.19 (1H, dd), 7.01 (1H, d), 6.57 (1H, d), 5.02 (1H, brs), 3.83 (2H, s).

MS: APCI (+ve) 262

### EXAMPLE 3

### 3-(5-Chlorobiphenyl-2-yl)propanoic acid

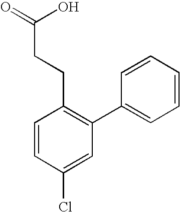

(i) 5-Chlorobiphenyl-2-carbaldehyde

The subtitle compound was prepared by the method of example 2 step (i) using 4-chloro-2-iodobenzaldehyde.

1H NMR CDCl₃: δ 9.92 (1H, s), 7.97 (1H, d), 7.51-7.35 (7H, m).

(ii) tert-Butyl (2E)-3-(5-chlorobiphenyl-2-yl)acrylate

Sodium hydride (60% wt. disp. oil, 0.2 g) was added to a solution of tert-butyl-P,P-dimethylphosphonoacetate (1.16 g) in dry DMF (20 ml) at 0-5° C. After 30 min, a solution of the product from step (i) (0.93 g) in DMF (5 ml) was added, and the mixture warmed to RT. After 3 h the mixture was quenched with water and extracted with diethylether. The organic layer was washed with water, dried, and the solvent evaporated under reduced pressure. The residue was purified by chromatography on silica eluting with 5% ethyl acetate/isohexane, yield 1.22 g.

MS: APCI (+ve) 259 (M+1-tBu)

(iii) 3-(5-Chlorobiphenyl-2-yl)propanoic acid

The product from step (ii) (0.35 g) and 10% Pd/C (0.05 g) in ethanol (10 ml) and ethyl acetate (10 ml) was hydrogenated at 2bar for 8 h then filtered through celite. The solvent was removed under reduced pressure and the residue dissolved in a mixture of DCM (10 ml) and trifluoroacetic acid (3 ml) and stirred at RT for 5 h. The solvent was removed under reduced pressure and the residue triturated with diethylether/isohexane then filtered, yield 0.045 g.

1H NMR CDCl₃: δ 7.45-7.20 (8H, m), 2.88 (2H, t), 2.41 (2H, t).

MS: APCI (−ve) 259/261

### EXAMPLE 4

### (2E)-3-(5-Chlorobiphenyl-2-yl)acrylic acid

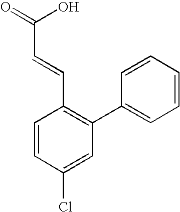

The product from example 3 step (ii) (0.25 g) was dissolved in DCM (10 ml) and trifluoroacetic acid (3 ml), stirred at RT for 4 h then evaporated under reduced pressure. The residue was triturated with diethylether/isohexane then filtered, yield 0.061 g.

1H NMR CDCl₃: δ 7.72 (1H, d), 7.65 (1H, d), 7.48-7.28 (7H, m), 6.36 (1H, d).

MS: APCI (−ve) 257/9

### EXAMPLE 5

### N-(5-Chlorobiphenyl-2-yl)-N-methylglycine

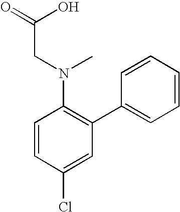

The product from example 2 step (ii) (1.2 g) and sodium hydrogencarbonate (0.77 g) in dimethylsulphate (5 ml) was heated at 90° C. for 2 h. The mixture was acidified with 2M hydrochloric acid, extracted with ethyl acetate, the organics were dried and evaporated under reduced pressure. The residue was purified by RPHPLC.

1H NMR DMSO-d6: δ 7.54-7.27 (6H, m), 7.13-7.06 (2H, m), 3.46 (2H, s), 2.66 (3H, s).

MS: ESI (−ve) 274

### EXAMPLE 6

### (5-Chlorobiphenyl-2-yl)acetic acid

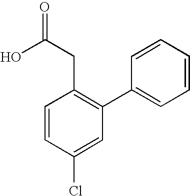

(i) 5-Chloro-2-(chloromethyl)biphenyl

Sodium borohydride (0.21 g) was added to a solution of the product from example 3 step (i) (1.15 g) in methanol (20 ml) and stirred at RT for 2 h. The mixture was quenched with water, extracted with diethylether, the organics dried and evaporated under reduced pressure. The residue was dissolved in DCM (20 ml) then thionyl chloride (0.45 ml) added and stirred at RT for 2 h. The solvent was removed under reduced pressure and the residue partitioned between diethylether and water. The organics were dried and evaporated under reduced pressure to give an oil, yield 1.5 g.

(ii) (5-Chlorobiphenyl-2-yl)acetic acid

The product from step (i) (1.5 g), sodium cyanide (0.32 g) and 18-crown-6 (catalytic) in acetonitrile (20 ml) were heated under reflux for 5 h. The mixture was partitioned between ethyl acetate and water, the organic layer dried, and the solvent evaporated under reduced pressure. The residue was dissolved in ethanol (5 ml) then potasium hydroxide (0.3 g) and water (15 ml) added and the mixture heated under reflux for 10 h. The mixture was acidified with 2M hydrochloric acid and extracted with ethyl acetate, the organics dried and evaporated under reduced pressure. The residue was purified by RPHPLC, yield 0.08 g.

1H NMR DMSO-d6: δ 12.32 (1H, bs), 7.48-7.26 (8H, m), 3.32 (2H, s).

MS: ESI(−ve) 245 (M−1)

### EXAMPLE 7

### {[5-Chloro-4′-(ethylthio)biphenyl-2-yl]thio}acetic acid

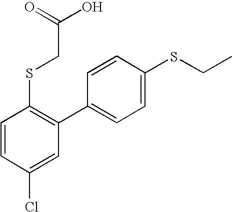

(i) 5-Chloro-4′-(ethylthio)biphenyl-2-amine

The subtitle compound was prepared by the method of example 2 step (i) using 4-(ethylthio)benzeneboronic acid

MS: ESI(+ve) 264

(ii) {[5-Chloro-4′-(ethylthio)biphenyl-2-yl]thio}acetic acid

A solution of sodium nitrite (0.735 g) in water (5 ml) was added to the product from step (i) (2.8 g) in conc. hydrochloric acid (12 ml) and water (30 ml) at 5° C. The mixture was stirred for 15min then thioglycolic acid (0.74 ml) was added, followed by a solution of sodium hydrogencarbonate (2.25 g) in water (25 ml). The mixture was heated at 100° C. for 1 h, diluted with water and extracted with diethylether. The aqueous layer was acidified with 2M hydrochloric acid, extracted with ethyl acetate and the organic layer dried and evaporated under reduced pressure. The residue was purified by RPHPLC, yield 0.025 g.

1H NMR DMSO-d6: δ 7.45-7.26 (7H, m), 3.74 (2H, s), 3.07-3.00 (2H, q), 1.30-1.27 (3H, t).

MS: ESI(−ve) 337 (M−1)

### EXAMPLE 8

### {[5-Chloro-4′-(ethylthio)biphenyl-2-yl]thio}acetic acid

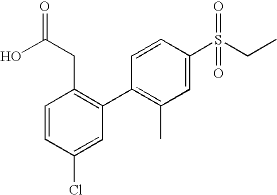

(i) 4-Bromo-3-methylphenyl ethyl sulfide

Bromine (2.2 ml) was added to a solution of 1-(ethylthio)-3-methylbenzene (6.6 g) in acetic acid (30 ml) at 0° C. The mixture was stirred at RT for 2 h then evaporated under reduced pressure. The residue was purified by chromatography on silica eluting with dichloromethane, yield 6.6 g.

1HNMR CDCl₃: δ 7.43-6.97 (3H, m), 2.97-2.87 (2H, q), 2.36 (3H, s), 1.40-1.27 (3H, t).

(ii) 2-[4-(Ethylthio)-2-methylphenyl]-4,4,5,5-tetramethyl-1,3,2-dioxaborolane

The product from step (i) (1 g), 4,4,5,5-tetramethyl-1,3,2-dioxaborolane (1.94 ml), triethylamine (2.4 ml) and [1,1′-bis(diphenylphosphino)ferrocene]palladium(II) chloride 1:1 complex with DCM (0.16 g) in dioxane (20 ml) was heated at 85° C. for lOh. The mixture was quenched with aqueous ammonium chloride solution and extracted with diethylether. The organics were dried, evaporated under reduced pressure and the residue purified by chromatography on silica eluting with 50% dichloromethane/isohexane, yield 0.7 g.

1HNNMR CDCl₃: δ 7.67-7.65 (1H, d), 7.08-7.05 (2H, m), 2.94-2.92 (2H, q), 2.50 (3H, s), 1.43-1.27 (15H, m).

(iii) (4-Chloro-2-iodophenyl)acetonitrile

4-Chloro-2-iodobenzylchloride (4 g), sodium cyanide (0.672 g) and 18-crown-6 (catalytic) in acetonitrile (40 ml) were heated under reflux for 20 h. The mixture was partitioned between diethylether and water, the organic layer dried, and the solvent evaporated under reduced pressure. The residue was purified by chromatography on silica eluting with diethylether/isohexane (1:5), yield 2 g.

1H NMR CDCl₃: δ 7.88-7.33 (3H, m), 3.79 (2H, s).

(iv) [5-Chloro-4′-(ethylthio)-2′-methylbiphenyl-2-yl]acetonitrile

The product from step (ii) (0.6 g), the product from step (iii) (0.59 g), cesium fluoride (0.69 g), [1,1′-bis(diphenylphosphino)ferrocene]palladium(II) chloride 1:1 complex with DCM (0.08 g) in dioxane (20 ml) was heated at 90° C. for 20 h. The mixture was partitioned between ethyl acetate and water, the organic layer dried, and the solvent evaporated under reduced pressure. The residue was purified by chromatography on silica eluting with 25% diethylether/isohexane, yield 0.6 g.

MS: ESI(−ve) 300 (M−1)

(v) {[5-Chloro-4′-(ethylthio)biphenyl-2-yl]thio}acetic acid

The product from step (iv) (0.6 g) and potassium hydoxide (0.135 g) in ethanol (10 ml) and water (10 ml) was heated under reflux for 72 h. The mixture was acidified with 2M hydrochloric acid and extracted with ethyl acetate, the organics were dried and evaporated under reduced presure. The residue was dissolved in DCM (10 ml), MCPBA (0.215 g) was added and the mixture stirred at RT for lh then evaporated under reduced pressure. The residue was purified by RPHPLC, yield 0.03 g.

1H NMR DMSO-d6: δ 7.84-7.25 (6H, m), 3.53-3.16 (4H, m), 2.10 (3H, s), 1.15-1.09 (3H, t).

MS: ESI(−ve) 351 (M−1)

### EXAMPLE 9

### N-[4′-(Ethylsulfonyl)-5-(trifluoromethyl)biphenyl-2-yl]glycine

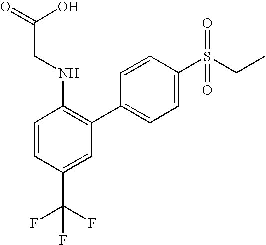

(i) 4′-(Ethylthio)-5-(trifluoromethyl)biphenyl-2-amine

The subtitle compound was prepared by the method of example 8 step (ii) using 4-(ethylthio)benzeneboronic acid and 2-iodo-4-trifluoromethylaniline.

MS: ESI(−ve) 272 (M−1)

(ii) 4′-(Ethylsulfonyl)-5-(trifluoromethyl)biphenyl-2-amine

MCPBA (18.8 g) was added to a solution of the product from step (i) (13 g) in DCM (lOOml) and stirred at RT overnight. The mixture was partitioned between DCM/aq. sodium metabisulphite solution, the organics washed with aq. sodium hydrogencarbonate solution, water, dried and evaporated under reduced pressure. Yield 9.0 g

1H NMR CDCl₃: δ 8.03-7.35 (6H, m), 6.83-6.80 (1H, d), 4.15-4.04 (2H, bs), 3.21-3.14 (2H, q), 1.36-1.31 (3H, t).

(iii) Ethyl N-[4′-(ethylsulfonyl)-5-(trifluoromethyl)biphenyl-2-yl]glycinate A solution of titanium tetrachloride (1M in DCM) (1.5 lml) was added to a mixture of the product from step (ii) (0.5 g), ethyl glyoxalate solution (0.3 ml) and triethylamine (0.21 ml) in DCM (10 ml) and stirred at RT for 1 h. The mixture was washed with water, dried and evaporated under reduced pressure. The residue was purified by chromatography on silica eluting with 80% diethylether/isohexane, yield 0.35 g.

1H NMR CDCl₃: δ 8.04-6.60 (7H, m), 4.77-4.73 (1H, t), 4.25-4.18 (2H, q), 3.93-3.91 (2H, d), 3.22-3.14 (2H, q), 1.37-1.23 (6H, 2xt).

(iv) N-[4′-(Ethylsulfonyl)-5-(trifluoromethyl)biphenyl-2-yl]glycine

A solution of sodium hydroxide (0.033 g) in water (10 ml) was added to a solution ofthe product from step (iii) (0.35 g) in methanol (10 ml) and stirred at RT overnight. The mixture was acidified with 2M hydrochloric acid and extracted with ethyl acetate. The organics were dried, evaporated under reduced pressure and the residue purified by RPHPLC, yield 0.13 g.

H NMR DMSO-d6: δ 7.99-7.29 (6H, m), 6.69-6.67 (111, d), 5.78 (1H, t), 3.69 (2H, m), 3.41-3.32 (2H, q), 1.17-1.14 (3H, t).

MS: ESI(−ve) 386 (M−1)

### EXAMPLE 10

### 3-[4′-(Ethylsulfonyl)-2′-methyl-5-(trifluoromethyl)biphenyl-2-yl]propanoic acid

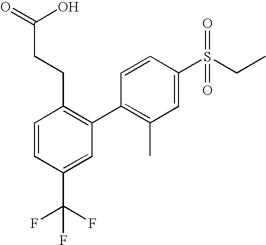

(i) Methyl 3-[2-bromo-4-(trifluoromethyl)phenyl]propanoate

3-[2-Bromo-4-(trifluoromethyl)phenyl]propanoic acid (2.04 g) was added to a preformed solution of acetyl chloride (0.48 ml) in methanol (30 ml) and stirred at RT for 2 h. The solvent was evaporated under reduced pressure, yield 1.8 g.

1H NMR CDCl₃: δ 7.80-7.26 (3H, m), 3.69-3.68 (3H, s), 3.15-3.10 (2H, t), 2.70-2.65 (2H, t).

(ii) Methyl 3-[4′-(ethylthio)-2′-methyl-5-(trifluoromethyl)biphenyl-2-yl]propanoate

The subtitle compound was prepared by the method of example 8 step (ii) using the product from step (i).

1H NMR CDCl₃: δ 7.81-7.01 (6H, m), 3.60 (3H, s), 3.02-2.96 (2H, q), 2.85-2.65 (2H, t), 2.42-2.38 (2H, t), 2.01 (3H, s), 1.38-1.20 (3H, t).

(iii) 3-[4′-(Ethylsulfonyl)-2′-methyl-5-(trifluoromethyl)biphenyl-2-yl]propanoic acid

The title compound was prepared by the method of example 9 steps (ii) and (iv) using the product from step (ii). Yield 0.186 g

1H NMR DMSO-d6: δ 7.87-7.37 (6H, m), 3.38-3.31 (2H, q), 2.63-2.46 (1H, m), 2.09 (3H, s), 2.04-1.96 (2H, t), 1.54-1.24 (1H, m), 1.21-1.05 (3H, t).

MS: ESI(−ve) 399 (M−1)

### EXAMPLE 11

### ({2-[4-(Ethylsulfonyl)-2-methylphenyl]-6-methylpyridin-3-yl}oxy)acetic acid

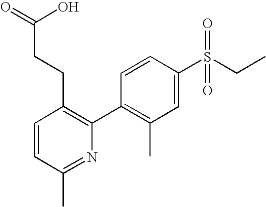

(i) tert-Butyl [(2-iodo-6-methylpyridin-3-yl)oxy]acetate

6-Iodo-2-picolin-5-ol (0.25 g), potassium carbonate (0.15 g) and tert-butyl bromoacetate (0.17 ml) in DMF (2 ml) was stirred at RT overnight. The reaction was quenched with water and extracted with ethyl acetate, the organics were dried and evaporated under reduced pressure, yield 0.28 g.

(ii) ({2-[4-(Ethylsulfonyl)-2-methylphenyl]-6-methylpyridin-3-yl } oxy)acetic acid The title compound was prepared by the methods of example 8 step (iv), example 9 step (ii) and example 4 using the product from step (i). Yield 0.175 g

¹H NMR CDCl₃: δ 7.80-7.73 (2H, m ),7.53 (1H, d), 7.42 (1H, d), 7.28 (1H, d), 4.73 (2H, s), 3.32 (2H, q), 2.48(3H, s), 2.13 (3H, s), 1.17 (3H, s) . MS: APCI(+ve) 350.

### EXAMPLE 12

### [2-(2-Thienyl)-4-(trifluoromethyl)phenoxy]acetic acid

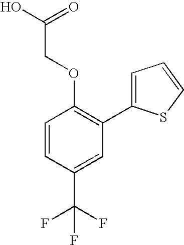

(iii) [4-(Trifluoromethyl)phenoxy]-acetic acid

Sodium hydride (2.96 g, 60% disp. in oil) was added to a stirred solution of 4-hydroxybenzo-trifluoride (10 g) in tetrahydrofuran (150 ml) at −78° C. The cooling bath was removed, the mixture stirred for lh then methyl bromoacetate (7 ml) added. After 1 h, water was added, the tetrahydrofuran evaporated off under reduced pressure and the residue partitioned between ethyl acetate/2M hydrochloric acid. The organic layer was evaporated under reduced pressure and the residue dissolved in tetrahydrofuran (120 ml). Methanol (30 ml), water (30 ml) and conc. sodium hydroxide solution (6 ml) was added and the mixture stirred at RT overnight. The organics were removed under reduced pressure and the residue partitioned between ethyl acetate and 2M hydrochloric acid. The organics were separated, dried and evaporated under reduced pressure, yield 12.42 g

¹H NMR DMSO-d6: δ 13.13 (1H, s), 7.65 (2H, d), 7.10 (2H, d), 4.80 (2H, s).

MS: APCI (−ve) 219 (M−1)

(i) [4-(Trifluoromethyl)phenoxy]-acetic acid, (3-methyl-3-oxetanyl)methyl ester

Oxalyl chloride (14 ml) was added to a solution of the product from step (i) (12.42 g) and N,N-dimethylformamide (2 drops) in dichloromethane (100 ml), and stirred at RT for 72 h. The mixture was evaporated under reduced pressure, the residue dissolved in dichloromethane (100 ml) then triethylamine (20 ml) and 3-methyl-3-oxetanemethanol (17 ml) added. After 2h the mixture was washed with water, evaporated under reduced pressure and the residue purified by chromatography on silica eluting with dichloromethane, yield 14.2 g.

¹H NMR DMSO-d6: δ 7.66 (2H, d); 7.14 (2H, d); 4.98 (2H, s), 4.34 (2H, d); 4.24 (2H, s); 4.19 (2H, d), 1.21 (3H, s).

(ii) 4-Methyl-1-[[4-(trifluoromethyl)phenoxy]methyl]-2,6,7-trioxabicyclo[2.2.2]octane

Boron trifluoride diethyl etherate (1.48 ml) was added to a solution of the product from step (ii) (14.2 g) in dichloromethane at −78° C. The cooling bath was removed, the mixture stirred for 3 h then triethylamine (6.2 ml) added. The mixture was reduced to half the volume under reduced pressure then filtered. The filtrate was evaporated under reduced pressure then the residue purified by chromatography on silica (pre-eluted with one column volume of neat triethylamine) eluting with dichloromethane, yield 11.1 g.

¹H NMR DMSO-d6: δ 7.62 (2H, d); 7.14 (2H, d); 4.04 (2H, s); 3.91 (6H, s); 0.77 (3H, s).

MS: APCI (+ve) 305 (M+1)

(ii) [2-Borono-4-(trifluoromethyl)phenoxy]-acetic acid

A solution of sec-butyllithium (66 ml, 1.4M in cyclohexane) was added dropwise over 10 min to a stirred solution of the product from step (iii) (9.44 g) in THF (100 ml) at −78° C. After 3 h the mixture was warmed to −40° C. for Smin, then cooled to −78° C. Trimethylborate (14.1 ml) was added, then after 1 Omin the reaction quenched with 2M hydrochloric acid. The mixture was warmed to RT and the organic phase separated. The aqueous layer was extracted with ethyl acetate, the organics combined and evaporated under reduced pressure. The residue was dissolved in methanol (500 ml) then bondelut-NH₂ resin(180 g) added and the mixture swirled for 0.5 h then filtered. The resin was washed with 10% acetic acid/methanol, the washings then evaporated under reduced pressure and dried under high vacuum. The residue was dissolved in methanol(50 ml), tetrahydrofuran (50 ml) and saturated aqueous sodium hydroxide solution (2 ml), left for 30 min then 2M hydrochloric acid (50 ml) added and the organics evaporated under reduced pressure. The residual aqueous layer was extracted with ethyl acetate, the organics separated, dried and evaporated under reduced pressure, yield 5.05 g.

¹H NMR DMSO-d6: δ 8.07 (1H, s); 7.89 (2H, d); 7.75 (2H, dd); 7.14 (1H, d); 4.85 (2H, s).

MS: APCI (−ve) 263 (M−1)

(ii) [2-(2-Thienyl)-4-(trifluoromethyl)phenoxy]acetic acid

A mixture of the product from step (iv) (0.1 g), 2-bromothiophene (0.12 g), tetrakis (triphenylphosphine)palladium(O) (0.046 g) and sodium carbonate (0.12 g) in methanol (2 ml) was heated in a CEM microwave (variable wattage up to 150 W) at 100° C. for 10 min. The mixture was loaded onto SCX resin (sulphonic acid resin), flushed with methanol then the product eluted with methanol/ammonia. The methanol/ammonia filtrate was evaporated under reduced pressure then loaded onto bondelut-NH₂ resin. The resin was flushed with methanol then the product eluted with methanol/acetic acid. The methanol/acetic acid filtrate was evaporated and the residue purified by RPHPLC. Yield 0.039 g

¹H NMR DMSO-d6: δ 7.96 (1H, d), 7.83 (1H, d), 7.64 (1H, d), 7.64 (1H, s), 7.23 (1H, d), 7.16 (1H, dd), 4.97 (2H, s)

MS: APCI (−ve) 301 (M−1)

### EXAMPLE 13-18

The following compounds were synthesised in an analogous method to example 12

### EXAMPLE 13

### [2-(2-Cyano-3-thienyl)-4-(trifluoromethyl)phenoxy]acetic acid

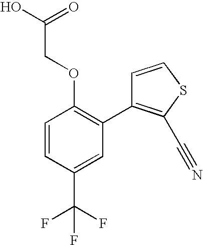

¹H NMR DMSO-d6: δ 8.08 (1H, d), 7.60 (1H, d), 7.47 (1H, s), 7.65 (1H, d), 7.19 (1H, d), 4.59, (2H, s)

### EXAMPLE 14

### [2-(2-Chloro-3-thienyl)-4-(trifluoromethyl)phenoxy]acetic acid

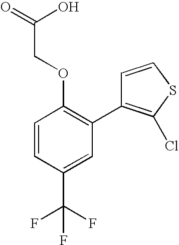

¹H NMR DMSO-d6: δ 7.77 (1H, d), 7.65 (1H, s), 7.49 (1H, d), 7.31 (1H, d), 7.11 (1H, d), 4.52 (2H, s)

MS: APCI (+ve) 335 (M+1)

### EXAMPLE 15

### [2-(2,5-Dichloro-3-thienyl)-4-(trifluoromethyl)phenoxy]acetic acid

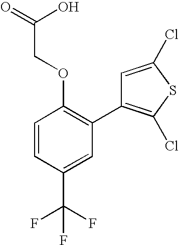

¹H NMR DMSO-d6: δ 7.70 (1H, d), 7.64 (1H, s), 7.41 (1H, s), 7.16 (1H, d), 4.62 (2H, s)

### EXAMPLE 16

### [2-(3-Thienyl)-4-(trifluoromethyl)phenoxy]acetic acid

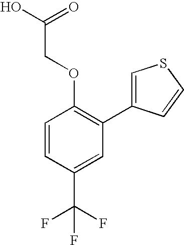

¹H NMR DMSO-d6: δ 8.13 (1H, s), 7.87 (1H, s), 7.63 (2H, d), 7.61 (1H, d), 7.19 (1H, d), 4.86 (2H, s)

MS: APCI (−ve) 301 (M−1)

### EXAMPLE 17

### [2-(5-Acetyl-2-thienyl)-4-(trifluoromethyl)phenoxy]acetic acid

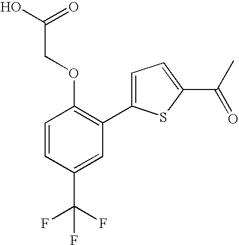

¹H NMR DMSO-d6: δ 8.33 (1H, d), 7.92 (1H, d), 7.57 (1H, s), 7.59 (1H, d), 7.28 (1H, d), 4.72 (2H, s), 2.50 (3H, s) (under DMSO)

MS: APCI (−ve) 343 (M−1)

### EXAMPLE 18

### [2-(2-Furyl)-4-(trifluoromethyl)phenoxy]acetic acid

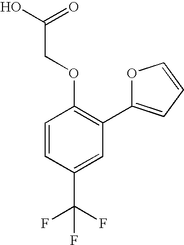

¹H NMR DMSO-d6: δ 8.92 (s, 1H), 7.90 (s, 1H), 7.74-7.71 (m, 1H), 7.52 (d, J=8.5 Hz, 1H), 7.25-7.21 (m, 1H), 7.09 (d, J=8.7 Hz, 1H), 4.42 (s, 2H)

MS: APCI (−ve) 285 (M−1)

### EXAMPLE 19

### (2S)-2-[4-(Trifluoromethyl)-2-(1,3,5-trimethyl-1H-pyrazol-4-yl)phenoxy]propanoic acid

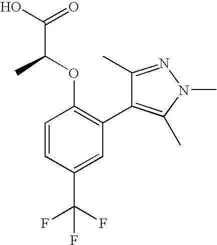

(i) Benzyl 2-bromo-4-(trifluoromethyl)phenyl ether

Benzyl bromide (7.44 ml) was added to a mixture of 2-bromo-4-trifluoromethylphenol (16.75 g) and potassium carbonate (9.6 g) in acetonitrile (250 ml). After stirring at RT overnight the solvent was removed under reduced pressure and the residue partitioned between isohexane/water. The organic layer was separated, washed with 1M sodium hydroxide solution, brine, dried and evaporated under reduced pressure to give an oil. Piperidine (2 ml) was added to the oil then the mixture was partitioned between diethylether/2M hydrochloric acid, the organics separated, washed with brine, dried and evaporated under reduced pressure, yield 15.6 g.

¹H NMR CDCl₃: δ 7.83 (1H, d); 7.53-7.30 (6H, m); 6.98 (1H, d); 5.22 (2H, s)

(ii) [2-(Benzyloxy)-5-(trifluoromethyl)phenyl]boronic acid

A solution of the product from step (i) (10 g in 15 ml THF, 5 ml), was added to a mixture of magnesium turnings in THF (10 ml). When the reaction had initiated the remainder of the solution was added at such a rate to keep the temperature at 50° C. After lh the solution was added to a solution of trimethylborate (6.8 ml) in THF (15 ml) at 0° C. The mixture was warmed to RT, stirred for 2 h then poured into water. The precipitate was filtered off and purified by chromatography on silica eluting with 20% ethyl acetate/isohexane, yield 4.5 g.

¹H NMR DMSO-d6: δ 8.02 (2H, s), 7.76 (1H, d), 7.70 (1H, m), 7.50 (2H, d), 7.41 (2H, m), 7.34 (1H, m), 7.21 (1H, d), 5.25 (2H, S)

(iii) 4,4,5,5-Tetramethyl-2-[2-(phenylmethoxy)-5-(trifluoromethyl)phenyl]-1,3,2-dioxaborolane

Pinacol (1.82 g) was added to a solution of the product from step (ii) (4.54 g) in diethylether (40 ml) and stirred at RT for 20 h. The reaction was diluted with diethylether (100 ml), washed with brine, dried (MgSO₄) and evaporated under reduced pressure. Yield 5.7 g.

¹H NMR DMSO-d6: δ 7.82 (d, 1H), 7.79 (d, 1H), 7.6 (d, 2H), 7.4 (t, 2H), 7.32 (d, 1H), 7.27 d, 1H), 5.24 (s, 2H), 1.32 (s, 12H)

(iv) 2-(4,4,5,5-Tetramethyl-1,3,2-dioxaborolan-2-yl)-4-(trifluoromethyl)phenol 10% Pd/C (0.5 g) was added to a solution of the product from step (iii) in EtOAc (80 ml) and ydrogenated at RT and 1 bar for 1 h, and for a firther 3 h at 3 bar. The catalyst was removed y filtration and the filtrate evaporated to leave a solid product. Yield 4.2 g.

¹H NMR DMSO-d6: δ 9.99 (d, 1H), 7.72 (s, 1H), 7.63 (d, 1H), 6.99 (d, 1H), 1.3 (d, 12H)

(v) 2-[2-(4,4,5,5-Tetramethyl-1,3,2-dioxaborolan-2-yl)-4-(trifluoromethyl) phenoxy]-(2S)-propanoic acid, 1,1-dimethylethyl ester

Diethyl azodicarboxylate (1.97 ml) was added to a stirred solution of the product from step (iv) (2.86 g), tert-butyl-R-lactate (1.46 g) and triphenylphosphine (2.62 g) in THF (50 ml) at 0° C. The mixture was warmed to RT, stirred at RT overnight then the solvent evaporated under reduced pressure. The residue was purified by chromatography on silica eluting with ethyl acetate/iso-hexane. Yield 4.0 g

(vi) 2-[2-Borono-4-(trifluoromethyl)phenoxy]-(2S)-propanoic acid

TFA (1 0 ml) was added to a solution of the product from step (v) (4.0 g) in DCM (100 ml) and stirred for 30 min. The solvent was evaporated and the residue dissolved in a mixture of 1M hydrochloric acid (30 ml) and acetonitrile (30 ml) After lh the mixture was evaporated to dryness, dissolved in 1M sodium hydroxide, washed with ether and adjusted to pH 2 with concentrated hydrochloric acid. The aqueous was then extracted with diethylether, the diethylether layer washed with brine, dried (MgSO₄) and evaporated under reduced pessure.

Yield 2.0 g. The crude material was carried forward to step (vii).

(vii) (2S)-2-[4-(Trifluoromethyl)-2-(1,3,5-trimethyl-1H-pyrazol-4-yl)phenoxy] propanoic acid

A mixture of the product from step (vi) (0.lg), 4-bromo-1,3,5-trimethyl-1H-pyrazole (0.068 g), tetrakis(triphenylphosphine)palladium(O) (0.05 g), potassium carbonate (0.2 g) in dioxane (2 ml) was heated in a CEM microwave (variable wattage up to 150 W) at 100° C. for 20 min. The solvent was evaporated and the residue purified by RPHPLC. Yield 0.005 g

¹H NMR DMSO-d6: δ 7.52 (1H, dd), 7.30 (1H, d), 6.95 (1H, d), 4.55 (1H, q), 3.68 (3H, s), 2.15 (3H, s), 2.04 (3H, s), 1.35 (3H, d)

MS: APCI (−ve) 341 (M−1)

### EXMAPLE 20

### (2S)-2-[2-[1-Methyl-3-(trifluoromethyl)-1H-pyrazol-5-yl]-4-(trifluoromethyl)phenoxy] propanoic acid

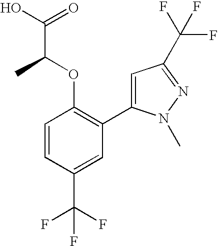

(i) 1-Methyl-3-(trifluoromethyl)-1H-pyrazol-5-yl trifluoromethanesulfonate Trifluoromethanesulfonic anhydride (1.15 ml) was added dropwise to a stirred solution of 1-methyl-4-(trifluoromethyl)-1H-pyrazole-3-ol (1.14 g) and triethylamine (0.50 ml) in DCM (25 ml) at 0° C. After 2 h, a further portion of trifluoromethanesulfonic anhydride (0.5 ml) was added, the DCM layer was washed with water, dried and evaporated under reduced pressure.

Yield 2 g

¹H NMR CDCl₃: δ 6.43 (1H, s), 3.90 (3H, s)

(ii) (2S)-2-[2-[1-Methyl-3-(trifluoromethyl)-1H-pyrazol-5-yl]-4-(trifluoromethyl) phenoxy]propanoic acid

The title compound was prepared by the method of example 19, using the product from step (i). Yield 0.015 g

¹H NMR DMSO-d6: δ 7.78 (1H, d), 7.64 (1H, d), 7.11 (1H, d), 6.86 (1H, s), 4.77 (1H, q), 3.88 (3H, s), 1.39 (3H, d)

MS: APCI (−ve) 381 (M−1)

### EXAMPLE 21

### (2S)-2-[2-[1-Methyl-5-(trifluoromethyl)-1H-pyrazol-3-yl]-4-(trifluoromethyl)phenoxy] ropanoic acid

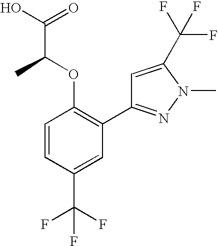

The title compound was prepared by the method of example 20, yield 0.03 g

¹H NMR DMSO-d6: δ 8.17 (1H, d), 7.64 (1H, dd), 7.53 (1H, s), 7.16 (1H, d), 4.99 (1H, q), 4.05 (3H, s), 1.54 (3H, d)

MS: APCI (−ve) 381 (M−1)

### EXAMPLE 22

### (2S)-2-[2-{1-[(Dimethylamino)sulfonyl]-3-methyl-1H-pyrazol-4-yl}-4-(trifluoromethyl)phenoxy]propanoic acid

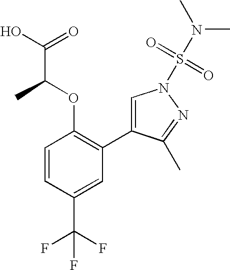

(i) 4-Bromo-N,N,3-trimethyl-1H-pyrazole-1-sulfonamide

A solution of potassium tert-butoxide (1M in THF, 18 ml) was added to a solution of 4-bromo-3-methylpyrazole (2.91 g) in THF (20 ml). The mixture was stirred at RT for 20 min then dimethylsulfamoyl chloride (1.94 ml) was added and stirred overnight. The solvent was evaporated under reduced pressure and the residue purified by chromatography on silica eluting with 20% ethyl acetate/isohexane.

¹H NMR DMSO-d6: δ 8.47 (1H, s), 2.85 (6H, s), 2.23 (3H, s)

(ii) (2S)-2-[2-{1-[(Dimethylamino)sulfonyl]-3-inethyl-1H-pyrazol-4-yl} -4-(trifluoromethyl)phenoxy]propanoic acid

The title compound was prepared by the method of example 19, using the product from step (i). Yield 0.05 g

¹H NMR DMSO-d6: δ 8.40 (1H, s), 7.64-7.60 (2H, m), 7.07 (1H, d), 4.83 (1H, q), 2.87 (6H), 2.31 (3H, s), 1.42 (3H, d)

MS: APCI (−ve) 420 (M−1)

### EXAMPLE 23

### {[2-(3-Cyanophenyl)pyridin-3-yl]oxy}acetic acid

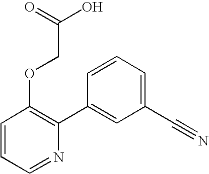

(i) Ethyl [(2-bromopyridin-3-yl)oxy]acetate

A mixture of 2-bromo-3-hydroxypyridine (1 g), potassium carbonate (0.873 g) and ethylbromoacetate (0.64 ml) in DMF (10 ml) was stirred at RT overnight then poured into water. The mixture was extracted with diethylether, the organics washed with brine, dried and evaporated under reduced pressure. Yield 1.4 g

¹H NMR DMSO-d6: δ 8.00 (1H, dd), 7.46 (1H, dd), 7.38 (1H, dd), 4.99 (2H, s), 4.17 (2H, q), 1.21 (3H, t)

(ii) {[2-(3-Cyanophenyl)pyridin-3-yl]oxy}acetic acid

A mixture of the product from step (i) (0.7 g), 3-cyanobenzeneboronic acid (0.39 g), tetrakis (triphenylphosphine)palladium(O), 2M sodium carbonate solution (8 ml) in ethanol (8 ml) and toluene (16 ml) was heated at 80° C. for 24 h. The mixture was partitioned between ethyl acetate/brine, the organics separated, dried and evaporated under reduced pressure. The residue was purified by RPHPLC.

¹H NMR DMSO-d6: δ 8.45 (1H, dd), 8.36-8.31 (2H, m), 7.87 (1H, dt), 7.67 (1H, dt), 7.59 (1H, dd), 7.43 (1H, dd), 4.90 (2H, s)

MS: ESI (−ve) 253

### EXAMPLE 24

### ({2-[2-Chloro-4-(methylsulfonyl)phenyl]-6-methylpyridin-3-yl}oxy)acetic acid

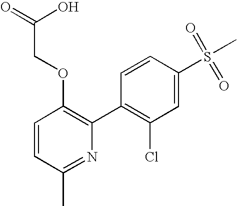

(i) [2-Chloro-4-(methylthio)phenyl]boronic acid

A solution of n-butyllithium (2.5M in hexanes, 390 ml) was added dropwise over 2.5 h to a solution of 4-bromo-3-chloromethylthiophenol (232 g) and tri-isopropylborate (224 ml) in THF (750 ml) and toluene (1 L) at −70° C. After stirring for lh, the mixture was allowed to warm to −30° C. then quenched with 2M hydrochloric acid (1 L). The mixture was stirred for 68 h, the organic layer separated and the aqueous layer extracted with tert-butyhnethylether. The organics were combined washed with water, dried and evaporated under reduced pressure to give a solid which was triturated with 2% tert-butylmethylether in isohexane. Yield 149 g

¹H NMR CDCl₃: δ 7.80 (1H, d); 7.16 (1H, s); 7.13 (1H, d); 6.35 (2H, s; 2.49 (3H, s)

(ii) 2-[2-Chloro-4-(methylthio)phenyl]-6-methylpyridin-3-ol

The subtitle compound was prepared by the method of example 23 step (ii), using the product from step (i) and 6-iodo-2-picolin-5-ol. Yield 0.4 g

¹H NMR DMSO-d6: δ 9.64 (1H, s), 7.34 (1H, d), 7.26 (2H, d), 7.18 (1H, d), 7.09 (1H, d), 2.37 (3H, s), 2.54 (3H, s)

(iii) Ethyl ({2-[2-chloro-4-(methylthio)phenyl]-6-methylpyridin-3-yl}oxy)acetate

The subtitle compound was prepared by the method of example 23 step (i), using the product from step (ii), yield 0.25 g

(iv) ({2-[2-Chloro-4-(methylsulfonyl)phenyl]-6-methylpyridin-3-yl}oxy)acetic acid

A solution of the product from step (iii) (0.25 g) and mcpba (0.28 g) in DCM (5 ml) was stirred at RT overnight, then the solvent removed under reduced pressure. The residue was dissolved in IM aqueous sodium hydroxide solution (1 ml) and THF and the mixture stirred at RT for 72 h. The mixture was purified by RPHPLC, yield 0.03 g.

¹H NMR DMSO-d6: δ 8.04 (1H, d), 7.93 (1H, dd), 7.75 (1H, d), 7.33 (1H, d), 7.25 (1H, d), 4.45 (2H, s), 3.34 (3H, s), 2.42 (3H, s)

MS: APCI (−ve) 354 (M−1)

### EXAMPLE 25

### (2S)-2-({2-[2-Chloro-4-(methylsulfonyl)phenyl]-6-methylpyridin-3-yl}oxy)propanoic acid

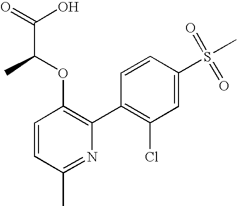

(i) 2-[2-Chloro-4-(methylsulfonyl)phenyl]-6-methylpyridin-3-ol

Trifluoroacetic acid (0.26 ml) was added to a solution of the product from example 24 step (ii) (0.447 g) in DCM (20 ml). After 5 min, mcpba (0.77 g) was added and the mixture stirred at RT overnight then washed with aqueous sodium hydrogencarbonate solution, brine, dried and evaporated under reduced pressure, yield 0.3 g.

¹H NMR DMSO-d6: δ 9.91 (1H, s), 8.04 (1H, d), 7.92 (1H, dd), 7.64 (1H, d), 7.25 (1H, d), 7.17 (1H, d), 3.34 (3H, s), 2.39 (3H, s)

(ii) (2S)-2-({2-[2-Chloro-4-(methylsulfonyl)phenyl]-6-methylpyridin-3-yl}oxy)propanoic acid

The title compound was prepared by the method of example 19 steps (v)-(vi), using the product from step (i), yield 0.26 g

¹H NMR DMSO-d6: δ 8.07 (1H, d), 7.96 (1H, dd), 7.73 (1H, d), 7.41 (1H, d), 7.33 (1H, d), 4.92 (1H, q), 3.35 (3H, s), 2.44 (3H, s), 1.38 (3H, d)

MS: APCI (−ve) 368 (M−1)

### EXAMPLE 26

### {[6-Amino-2-(3-cyanophenyl)pyridin-3-yl]oxy}acetic acid

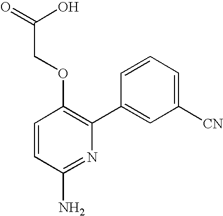

(i) 3-(6-Amino-3-methoxypyridin-2-yl)benzonitrile

The subtitle compound was prepared by the method of example 23 step (ii), using 6-bromo-5-methoxy-2-pyridinamine and 3-cyanophenylboronic acid, yield 0.3 g

¹H NMR DMSO-d6: δ 8.25 (1H, m), 8.23 (1H, ddt), 7.79 (1H, dt), 7.62 (1H, td), 7.41 (1H, d), 6.53 (1H, d), 5.68 (2H, s), 3.74 (3H, s)

(ii) 3-(6-Amino-3-hydroxypyridin-2-yl)benzonitrile

Boron tribromide (1M in DCM, 4.6 ml) was added to a solution of the product from step (i) (0.26 g) in DCM (10 ml) at 0° C., warmed to RT, stirred for 5 days then heated under reflux for 3 h, cooled and quenched with ice. The precipitate formed was filtered off and dried, yield 0.14 g.

¹H NMR DMSO-d6: δ 9.25 (1H, s), 8.42-8.37 (2H, m), 7.74 (1H, dt), 7.60 (1H, td), 7.14 (1H, d), 6.44 (1H, d), 5.47 (2H, s)

(iii) {[6-Amino-2-(3-cyanophenyl)pyridin-3-yl]oxy}acetic acid

The title compound was prepared by the method of example 19 steps (v)-(vi), using the product from step (ii).

¹H NMR DMSO-d6: δ 8.36 (1H, t), 8.24 (1H, dt), 7.89 (1H, dt), 7.72-7.60 (2H, m), 6.73 (1H, d), 4.72 (2H, s)

### EXAMPLE 27

### N-[4-Chloro-2-(5-methoxy-1H-indol-1-yl)phenyl]glycine

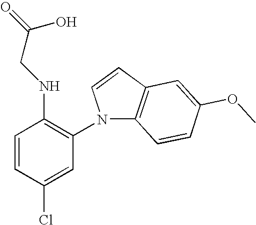

(i) 1-(5-Chloro-2-nitrophenyl)-5-methoxy-1H-indole

A mixture of 4-chloro-2-fluoro-nitrobenzene (1.19 g), 5-methoxyindole (1 g) and potassium carbonate (0.94 g) in NMP (7 ml) was heated at 60° C. for 4 h then 70° C. for 16 h. The mixture was partitioned between diethylether/water, the organics separated, washed with water, 2M sodium hydroxide solution, brine, dried and evaporated under reduced pressure. Yield 1.48 g

¹H NMR DMSO-d6: δ 8.22 (1H, d), 7.93 (1H, d), 7.80 (1H, dd), 7.49 (1H, d), 7.16 (1H, d), 7.02 (1H, d), 6.80 (1H, dd), 6.65 (1H, d), 3.78 (3H, s).

(ii) 4-Chloro-2-(5-methoxy-1H-indol-1-yl)aniline

The product from step (i) (1.48 g) and iron powder (1.5 g) in acetic acid was stirred at RT for 16 h then filtered through celite and the solvent removed under reduced pressure. The residue was partitioned between ethyl acetate/aqueous potassium carbonate solution, the organics separated, dried and evaporated under reduced pressure, yield 0.838 g.

¹H NMR DMSO-d6: δ 7.36 (1H, d), 7.21 (1H, dd), 7.15 (1H, d), 7.10 (1 H, d), 6.92 (2H, m), 6.79 (1H, dd), 6.59 (1H, d), 4.91 (2H, s), 3.77 (3H, s).

(iii) Ethyl N-[4-chloro-2-(5-methoxy-1H-indol-1-yl)phenyl]glycinate

The product from step (ii) (0.838 g), sodium acetate (0.76 g) and ethylbromoacetate (0.51 ml) in ethanol (6 ml) was heated under reflux for 24 h. The mixture was partitioned between ethyl acetate/water, the organics separated, washed with brine, dried and evaporated under reduced pressure. The residue was purified by chromatography on silica eluting with 10-20% ethyl acetate/isohexane.

¹H NMR DMSO-d6: δ 7.38 (1H, d), 7.34 (1H, dd), 7.17 (2H, m), 7.00 (1H, d), 6.80 (1H, dd), 6.76 (1H, d), 6.65 (1H, d), 4.99 (1H, t), 4.09 (2H, q), 3.92 (2H, d), 3.79 (3H, s), 1.18 (3H, t).

(iv) N-[4-Chloro-2-(5-methoxy-1H-indol-1-yl)phenyl]glycine

The product from step (iii) in THF (3 ml) and 2M aqueous sodium hydroxide (4 ml) was stirred at RT for 3 h then partitioned between diethylether/water. The aqueous layer was acidified and extracted with ethyl acetate, the organic extract was dried and evaporated under reduced pressure. The residue was purified by RPHPLC, yield 0.13 g.

¹H NMR DMSO-d6: δ 7.39 (1H, d), 7.34 (1H, dd), 7.18 (1H, d), 7.17 (1H, d), 7.01 (1H, d), 6.79 (1H, dd), 6.76 (1H, d), 6.65 (1H, dd), 4.86 (1H, t), 3.84 (2H, d), 3.79 (3H, s).

MS: APCI (−ve) 329 (M−1)

### EXAMPLE 28

### N-[4-Chloro-2-(5-cyano-1H-indol-1-yl)phenyl]glycine

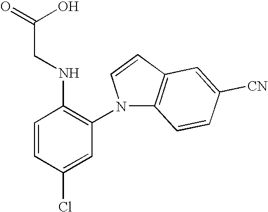

The title compound was prepared by the method of example 27, yield 0.102 g.

¹H NMR DMSO-d6: δ 8.22 (1H, d), 7.66 (1H, d), 7.48 (1H, dd), 7.35 (1H, dd), 7.22 (1H, d), 7.18 (1H, d), 6.86 (1H, dd), 6.70 (1H, d), 4.85 (1H, s), 3.29 (2H, s).

MS: APCI (−ve) 324 (M−1)

### EXAMPLE 29

### N-{4-Chloro-2-[5-(methylsulfonyl)-1H-indol-1-yl]phenyl}glycine

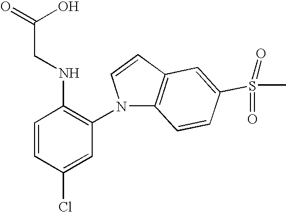

The title compound was prepared by the method of example 27, yield 0.12 g.

¹H NMR DMSO-d6: δ 12.65 (1H, s), 8.28 (1H, d), 7.66 (1H, m), 7.65 (1H, d), 7.38 (1H, dd), 7.29 (1H, d), 7.23 (1H, d), 6.95 (1H, dd), 6.78 (1H, d), 5.06 (1H, t), 3.81 (2H, d), 3.18 (3H, s).

MS: APCI (−ve) 377 (M−1)

### EXAMPLE 30

### [(5-Chloro-3′-cyanobiphenyl-2-yl)thio]acetic acid

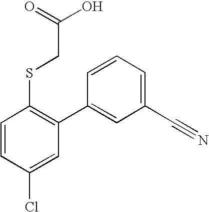

(i) [(2-Bromo-4-chlorophenyl)thio]acetic acid

Thioglycolic acid (0.43 ml) was added dropwise to a mixture of sodium hydride (60% in oil, 0.5 g) in DMSO (5 ml), after 30min 2-bromo-4-chloro-1-fluorobenzene (0.58 ml) was added and the mixture heated at 100° C. for 3 h. After cooling, water was added and the mixture extracted with diethylether. The organics were dried and evaporated under reduced pressure, yield 0.957 g.

¹H NMR DMSO-d6: δ 7.76 (1H, d), 7.48 (1H, dd), 7.31 (1H, d), 3.93 (2H, s).

(ii) Methyl [(2-bromo-4-chlorophenyl)thio]acetate

(Trimethylsilyl)diazomethane (2M in diethylether, 2 ml) was added to a solution of the product from step (i) (0.68 g) in methanol (5 ml) and stirred at RT for 10 min. The solvent was removed under reduced pressure to give an oil, yield 0.64 g.

¹H NMR DMSO-d6: δ 7.77 (1H, d), 7.47 (1H, d), 7.33 (1H, dd), 4.05 (2H, s), 3.66 (3H, s).

(iii) [(5-Chloro-3′-cyanobiphenyl-2-yl)thio]acetic acid

The title compound was prepared by the method of example 8 step (ii) and example 27 step (iv) using the product from step (ii) and 3-cyano phenylboronic acid, yield 0.028 g.

¹H NMR DMSO-d6: δ 12.82 (1H, bs), 7.90 (2H, m), 7.76 (1H, dt), 7.67 (1H, t), 7.49 (2H, d), 7.38 (1H, t), 3.77 (2H, s).

MS: APCI (−ve) 302 (M−1)

### EXAMPLE 31

### 3-[4′-(Methylsulfonyl)-3′,5-bis(trifluoromethyl)biphenyl-2-yl]propanoic acid

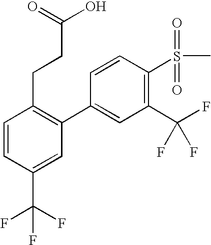

(i) Methyl 3-[4′-(methylthio)-3′,5-bis(trifluoromethyl)biphenyl-2-yl]propanoate

The subtitle compound was prepared by the methods of example 8 step (ii) and example 9 step (ii) using the product of example 10 step (ii) and 4,4,5,5-Tetramethyl-2-[4-(methylthio)-3-(trifluoromethyl)phenyl]-1,3,2-dioxaborolane [WO2004089885A1].

¹H NMR CDCl₃: δ 8.42 (1H, d), 7.86 (1H, s), 7.75 (1H, d), 7.67 (1H, d), 7.49 (1H, d), 7.45 (1H, s), 3.63 (3H, s), 3.27 (3H, s), 2.93 (2H, t), 2.52 (2H, t)

(ii) 3-[4′-(Methylsulfonyl)-3′,5-bis(trifluoromethyl)biphenyl-2-yl]propanoic acid

The title compound was prepared by the method of example 9 step (iv) using the product of step (i).

¹H NMR DMSO-d6: δ 12.16 (1H, s), 8.32 (2H, d), 8.05 (1H, s), 8.01 (1H, d), 7.79 (1H, d), 7.68 (1H, d), 3.38 (3H, s), 2.82 (2H, t), 2.52 (2H, t).

MS: APCI (−ve) 439 (M−1)

### EXAMPLE 32

### 3-(5-Chloro-3′-cyanobiphenyl-2-yl)propanoic acid

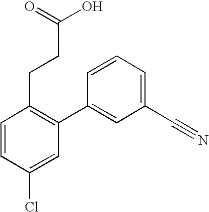

(i) (4-Chloro-2-iodobenzyl)malonic acid

4-chloro-2-iodotoluene (16.5 g), N-Bromosuccinimide (12.5 g), benzoyl peroxide (0.5 g) and ethyl acetate (300 ml) were charged to a flask and irradiated with a halogen lamp for 4h, cooled and the solvent removed under reduced pressure. Iso-haxane (300 ml) was added and the resulting solid was filtered. The filtrate was evaporated under reduced pressure. DMF (20 ml) was added and the solution added to a mixture of dimethylmalonate sodium salt [prepared from dimethylmalonate (10.7 ml) added drop wise to sodium hydride (60% weight, 2.8 g) in DMF (80 ml), stirred lh]. After stirring for lh the mixture was quenched with 2M HCl and partitioned between ether and water. The organics were separated, washed with water and evaporated under reduced pressure. The residue was dissolved in methanol (40 ml)/THF (40 ml) then 2M sodium hydroxide (100 ml) and stirred overnight, then stirred at reflux for 6 h. The solvent was evaporated under reduced pressure and the residue was extracted with ether. The aqueous layer was acidified 2M HCl and extracted with ethyl acetate. The organics were dried and evaporated under reduced pressure, yield 14.4 g.

MS: ESI (−ve) 353 (M−1)

(ii) 3-(4-Chloro-2-iodophenyl)propanoic acid, methyl ester

The product of step (i) (14.4 g) was heated at 130° C. for 2 h then cooled to room temperature. Methanol (250 ml) was added followed by trimethylsilyl chloride (30 ml) and the mixture stirred for 18 h at room temperature. The solvent was evaporated under reduced pressure and the residue was purified by chromatography on silica eluting with 5% ethyl acetate/isohexane, yield 9.84 g.

¹H NMR CDCl₃: δ 7.8 (1H, s), 7.26 (1H, d), 7.17 (1H, d), 3.68 (3H, s), 3.02 (2H, t), 2.61 (2H, t).

(iii) Methyl 3-(5-chloro-3′-cyanobiphenyl-2-yl)propanoate

The subtitle compound was prepared by the method of example 8 step (iv) using the product of step (ii) and 3-cyano phenylboronic acid.

¹H NMR CDCl₃: δ 7.71-7.68 (1H, m), 7.63-7.52 (3H, m), 7.32 (1H, d), 7.24 (1H, d), 7.18 (1H, s), 3.61 (3H, s), 2.84 (2H, t), 2.41 (2H, t).

(iv) 3-(5-Chloro-3′-cyanobiphenyl-2-yl)propanoic acid

The title compound was prepared by the method of example 9 step (iv) using the product of step (iii).

¹H NMR DMSO-d6: δ 12.10 (1H, s), 7.91-7.86 (2H, m), 7.73-7.64 (2H, m), 7.46-7.4 (2H, m), 7.28 (1H, s), 2.72 (2H, m), 2.38 (2H, t).

MS: APCI (−ve) 439 (M−1)

### EXAMPLE 33

### 3-[2′,5-Dichloro-4′-(methylsulfonyl)biphenyl-2-yl]propanoic acid

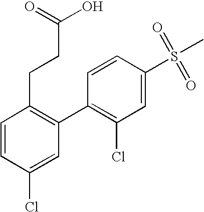

(i) Methyl 3-[2′,5-dichloro-4′-(methylthio)biphenyl-2-yl]propanoate

The subtitle compound was prepared by the method of example 8 step (ii) using the product of example 32 step (iii) and [2-chloro-4-(methylthio)phenyl]boronic acid [WO2004089885A1].

¹H NMR CDCl₃: δ 7.32-7.29 (2H, m), 7.25 (1H, d), 7.21 (1H, d), 7.17 (1H, s), 7.13-7.11 (1H, m), 3.6 (3H, s), 2.82-2.65 (2H, m), 2.53 (3H, s), 2.41 (2H, t).

(ii) Methyl 3-[2′,5-dichloro-4′-(methylsulfonyl)biphenyl-2-yl]propanoate

The subtitle compound was prepared by the method of example 9 step (ii) -using the product of step (i)

MS: ESI (+ve) 357 (M−OMe)

(iii) 3-[2′,5-Dichloro-4′-(methylsulfonyl)biphenyl-2-yl]propanoic acid

The title compound was prepared by the method of example 9 step (iv) using the product of step (ii).

¹H NMRDMSO-d6: δ 12.12 (1H, s), 8.14 (1H, s), 7.98 (1H, d), 7.66 (1H, d), 7.5-7.44 (2H, m), 7.24 (1H, s), 3.37 (3H, s), 2.68-2.35 (4H, m).

MS: APCI (−ve) 371 (M−1)

### EXAMPLE 34

### 3-[3′-Fluoro-4′-(pyrrolidin-1-ylcarbonyl)-5-(trifluoromethyl)biphenyl-2-yl]propanoic acid

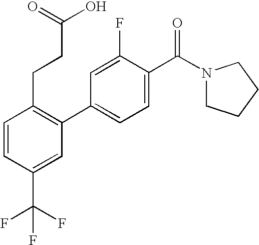

(i) 1-(4-Bromo-2-fluorobenzoyl)pyrrolidine

Oxalyl chloride (0.65 ml) was added to a solution of 4-bromo-2-fluorobenzoic acid (1.5 g) in DCM (20 ml), followed by DMF (catalytic amount). The mixture was stirred for 1 h, then added toluene and evaporated under reduced pressure. The residue was redissolved in DCM (20 ml) and pyrrolidine (1.2 ml) was added and then stirred overnight. The mixture was partitioned between water and DCM, dried and the solvents evaporated to give the subtitle compound, yield 2 g.

(ii) 2-Fluoro-4-(4,4,5,5-tetramethyl-1,3,2-dioxaborolan-2-yl)benzoic acid

Tricyclohexylphosphine (2.21 g), tris(dibenzylideneacetone)palladium (0) (0.34 g) and dioxan (30 ml) were charged to a flask and stirred for 30 min. Bis(pinacolato)diborane (1.96 g), the product from step (i) and potassium acetate (1.12 g) were added and the mixture stirred for 16 h. The mixture was partitioned between water and ethyl acetate, dried and the solvents evaporated. The residue was purified by chromatography on silica eluting with 50% ethyl acetate/isohexane, yield 9.84 g.

MS: ESI (+ve) 320 (M+1)

(iii) 3-[3′-Fluoro-4′-(pyrrolidin-1-ylcarbonyl)-5-(trifluoromethyl)biphenyl-2-yl]propanoic acid

The product of example 10 step (ii) (280 mg), the product of step (ii) (290mg), [1,1′-bis(diphenylphosphino)ferrocene]palladium(II) chloride (33 mg), sodium carbonate (190 mg) and dioxane (10 ml) were charged to a flask and heated at 90° C. for 16 h. The reaction mixture was diluted with 2M HCl, extracted with ethyl acetate, dried and the solvents evaporated under reduced pressure. The residue was purified by reverse phase HPLC to give the title compound, yield 30 mg.

¹H NMR DMSO-d6: δ 7.78-7.26 (6H, m), 3.52-3.48 (2H, t), 2.88-2.83 (2H, t), 3.25-3.17 (2H, t), 2.44-2.38 (2H, t), 1.99-1.82 (4H, m).

MS: APCI (−ve) 408 (M−1)

### EXAMPLE 35

### 3-[2′-Chloro-4-(methylsulfonyl)-5-(trifluoromethyl)biphenyl-2-yl]propanoic acid

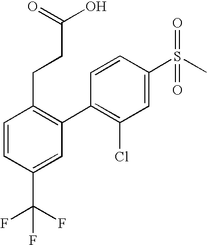

The title compound was prepared by the method of example 8 step (iv) and the method of example 9 step (iv) using the product of example 10 step (ii) and [2-chloro-4-(methylthio)phenyl]boronic acid [WO2004089885A1].

¹H NMR DMSO-d6: δ 12.31 (1H, s), 8.165-8.16 (1H, s), 8.0 (1H, d), 7.8-7.66 (3H, m), 7.52 (1H, s), 3.37 (3H, s), 2.76-2.55 (2H, m), 2.46-2.32 (2H, t).

MS: APCI (−ve) 405 (M−1)

### EXAMPLE 36

### 3-[4′-(Ethylsulfonyl)-3′,5-bis(trifluoromethyl)biphenyl-2-yl]propanoic acid

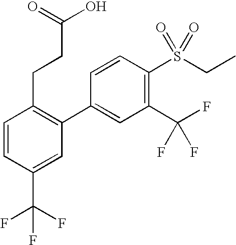

(i) 4-Bromo-1-(ethylthio)-2-(trifluoromethyl)-benzene

A mixture of sodium thioethoxide (3.64 g) and 5-bromo-2-fluorobenzotrifluoride (7.71 g) in DMF (15 ml) was heated at 50° C. for 1.5 h then poured into water and extracted with ether. The organics were dried and evaporated under reduced pressure. Yield 6.78 g

¹H NMR DMSO-d6: δ 7.52 (1H, d); 7.34 (1H, dd); 7.11 (1H, d); 2.93 (2H, q), 1.33 (3H, t).

(ii) [4-(Ethylthio)-3-(trifluoromethyl)phenyl]-boronic acid

n-BuLi (10 ml, 1.9M in hexane) was added drop wise to the product of step (i) (6.65 g) and tri-isopropyl borate (6 ml) in THF (20 ml) at −78° C. Stirred for 1 h, then quenched with aqueous HCl (16 ml) and extracted with diethyl ether. The organic layers were dried and concentrated under reduced pressure. The residue was purified by chromatography on silica eluting with 50% petrol/ether, yield 1.76 g.

¹H NMR DMSO-d6: δ 8.17 (1H, s), 7.89-7.69 (2H, m), 3.04 (2H, q), 1.03 (3H, t).

(iii) 3-[4′-(Ethylsulfonyl)-3′,5-bis(trifluoromethyl)biphenyl-2-yl]propanoic acid

The title compound was prepared by the method of example 8 step (iv) and the method of example 9 step (iv) using the product of example 10 step (ii) and the product of step (ii).

¹H NMR DMSO-d6: δ 8.26-8.24 (1H, d), 8.06 (2H, t), 7.72-7.58 (3H, m), 3.46-3.41 (2H, t), 2.74-2.67 (2H, t), 2.12-2.08 (2H, t), 1.23-1.19 (3H, t).

MS: APCI (−ve) 453 (M−1)

### EXAMPLE 37

### 3-[3′-Cyano-5′-fluoro-5-(trifluoromethyl)biphenyl-2-yl]propanoic acid

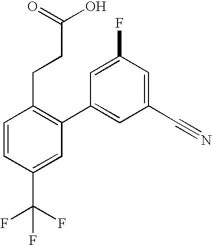

The title compound was prepared by the method of example 8 step (iv) and the method of example 9 step (iv) using the product of example 10 step (ii) and (3-cyano-5-fluorophenyl)boronic acid.

¹H NMR DMSO-d6: δ 12.31 (1H, s), 8.06-8.00 (2H, m), 7.79-7.77 (1H, d), 7.67-7.58 (3H, m), 2.7-2.66 (2H, t), 2.45-2.42 (2H, t).

MS: APCI (−ve) 336 (M−1)

### EXAMPLE 38

### 3-[3′-Cyano-5-(trifluoromethyl)biphenyl-2-yl]propanoic acid

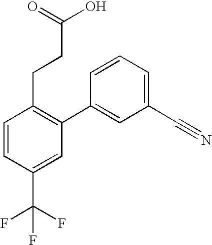

The title compound was prepared by the method of example 8 step (iv) and the method of example 9 step (iv) using the product of example 10 step (ii) and (3-cyanophenyl)boronic acid.

¹H NMR DMSO-d6: δ 12.32 (1H, s), 7.91-7.89 (2H, m), 7.75-7.54 (4H, m), 7.53 (1H, s), 2.7-2.83-2.79 (2H, t), 2.44-2.33 (2H, t).

MS: APCI (−ve) 318 (M−1)

### EXAMPLE 39

### 3-[5-Chloro-3′-fluoro-4′-(phenylsulfonyl)biphenyl-2-yl]propanoic acid

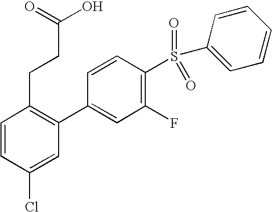

(i) 4-Bromo-2-fluoro-1-(phenylthio)benzene

Isoamylnitrite (2.02 ml) was added to a mixture of 4-fluoro-2-chloroaniline (2 g), diphenyldisulfide (2.22 g) in acetonitrile (30 ml), stirred at 60° C. for 2 h. The reaction mixture was concentrated to give the subtitle compound, yield 1.04 g.

¹H NMR CDCl₃: δ 7.5-7.38 (4H, m), 7.26-7.21 (3H, m), 7.08-6.9 (1H, m).

(ii) 4-Bromo-2-fluoro-1-(phenylsulfonyl)benzene

MCPBA (4.17 g) was added to a solution of the product from step i) (1.04 g) in dichloromethane (20 ml) and stirred for 2 h. The reaction mixture was washed with a solution of aqueous sodium metabisulfite, then aqueous sodium hydrogen carbonate. The organic layer was dried then concentrated under reduced pressure to give the subtitle compound, yield 1.15 g

¹H NMR CDCl₃: δ 7.5-7.38 (4H, m), 7.26-7.20 (3H, m), 7.02 (1H, m).

(iii) 3-[5-Chloro-3′-fluoro-4′-(phenylsulfonyl)biphenyl-2-yl]propanoic acid

The title compound was prepared by the method of example 8 step (iv) and the method of example 9 step (iv) using the product of example 32 step (ii) and the product of step (ii).

¹H NMR DMSO-d6: δ 8.12-8.06 (1H, t), 8.01-7.93 (2H, m), 7.8-7.62 (3H, m), 7.51-7.29 (4H, m), 7.14-7.13 (1H, s), 2.73-2.69 (2H, t), 2.40-2.63 (2H, t).

MS: APCI (−ve) 417 (M−1).

### EXAMPLE 40

### 3-[5-Chloro-4′-(pyridin-2-ylsulfonyl)biphenyl-2-yl]propanoic acid

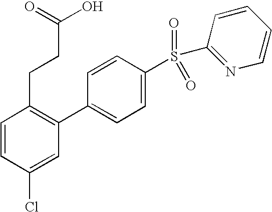

(i) 2-[(4-Bromophenyl)thio]pyridine

Sodium hydride (60% wt. 0.211 g) was added to 4-bromobenzenethiol (1 g) in DMF (20 ml) and stirred for 30 min, and then 2-chloropyridine (0.5 ml) was added. The reaction mixture was stirred for 20 h at 80° C. The reaction mixture was diluted with 2M sodium hydroxide, extracted with ethyl acetate, and then the organics were dried and evaporated under reduced pressure. The residue was purified by chromatography on silica eluting with 50% ether/isohexane, yield 1 g.

(ii) 2-[(4-Bromophenyl)sulfonyl]pyridine

The subtitle compound was prepared by the method of example 39 step (ii) using the product of step (i).

MS: ESI (+ve) 299 (M+H).

(iii) 3-[5-Chloro-4′-(pyridin-2-ylsulfonyl)biphenyl-2-yl]propanoic acid

The product of step (ii) (0.6 g), tetrakispalladiumtriphenylphosphine(0) (0.23 g), is(pinacolato)diboron (1 g), potassium acetate (0.88 g) and DMF (20 ml) were charged to a lask and stirred at 90° C. for 4 h. The product of example 9 step (iv) (0.6 g) and cesium fluoride (1 molar equivalent) were added and stirred at 90° C. for 8 h. The reaction was cooled and diluted with water, then extracted with ethyl acetate, dried and evaporated under reduced ressure. The residue was purified by chromatography on silica eluting with diethylether. he product of which was dissolved in 6N HCl (20 ml) and heated at 80° C. for 4 h and then quenched with sodium hydrogencarbonate solution. Extracted with ethyl acetate, dried and evaporated. The residue was purified by reverse phase HPLC to give the title compound, yield 0.1 g.

¹H NMR DMSO-d6: δ 8.74-8.72 (1H, m), 8.27-8.24 (1H, d), 8.19-8.15 (1H, t), 8.0)4-8.01 (2H, d), 7.69-7.63 (1H, m), 7.61 (2H, d), 7.44-7.43 (2H, m), 7.41 (1H, s), 2.71-2.65 (2H, t), 2.37-2.32 (2H, t).

MS: APCI (−ve) 400 (M−1).

Pharmacological Data

Ligand Binding Assay

[hu 3H]PGD₂ was purchased from Perkin Elmer Life Sciences with a specific activity of 100-210 Ci/mmol. All other chemicals were of analytical grade.

HEK cells expressing rhCRTh2/Gα16 were routinely maintained in DMEM containing 10% Foetal Bovine Serum (HyClone), 1 mg/ml geneticin, 2 mM L-glutamine and 1% non-essential amino acids. For the preparation of membranes, the adherent transfected HEKcells were grown to confluence in two layer tissue culture factories (Fisher, catalogue number TKT-170-070E). Maximal levels of receptor expression were induced by addition of 500 mM sodium butyrate for the last 18 hours of culture. The adherent cells were washed once with phosphate buffered saline (PBS, 50 ml per cell factory) and detached by the addition of 50 ml per cell factory of ice-cold membrane homogenisation buffer [20 mM HEPES (pH 7.4), 0.1 mM dithiothreitol, 1 mM EDTA, 0.1 mM phenyl methyl sulphonyl fluoride and 100 μg/ml bacitracin]. Cells were pelleted by centrifugation at 220×g for 10 minutes at 4° C., re-suspended in half the original volume of fresh membrane homogenisation buffer and disrupted using a Polytron homogeniser for 2×20 second bursts keeping the tube in ice at all times. Unbroken cells were removed by centrifugation at 220×g for 10 minutes at 4° C. and the membrane fraction pelleted by centrifugation at 90000×g for 30 minutes at 4° C. The final pellet was re-suspended in 4 ml of membrane homogenisation buffer per cell factory used and the protein content determined. Membranes were stored at −80° C. in suitable aliquots.

All assays were performed in Corning clear bottomed, white 96-well NBS plates (Fisher). Prior to assay, the HEK cells membranes containing CRTh2 were coated onto SPA PVT WGA beads (Amersham). For coating membranes were incubated with beads at typically 25 μg membrane protein per mg beads at 4° C. with constant agitation overnight. (The optimum coating concentrations were determined for each batch of membranes) The beads were pelleted by centrifugation (800×g for 7minutes at 4° C.), washed once with assay buffer (50 mM HEPES pH 7.4 containing 5 mM magnesium chloride) and finally re-suspended in assay buffer at a bead concentration of 10 mg/ml.

Each assay contained 20 μl of 6.25 nM [³H]PGD₂, 20 μl membrane saturated SPA beads both in assay buffer and 10 μl of compound solution or 13,14-dihydro-15-keto prostaglandin D₂ (DK-PGD₂, for determination of non-specific binding, Cayman chemical company). Compounds and DK-PGD₂ were dissolved in DMSO and diluted in the same solvent to 100× the required final concentration. Assay buffer was added to give a final concentration of 10% DMSO (compounds were now at 10× the required final concentration) and this was the solution added to the assay plate. The assay plate was incubated at room temperature for 2 hours and counted on a Wallac Microbeta liquid scintillation counter (1 minute per well). Compounds of formula (I) have an IC₅₀ value of less than (<) 10 μM.

Specifically, example 11 has a pIC₅₀=7.2, example 22 has a pIC₅₀=8.0, and example 31 has a pIC₅₀=7.4.

1. A compound of foumula (I) or a pharmaceutically acceptable salt or solvate thereof: 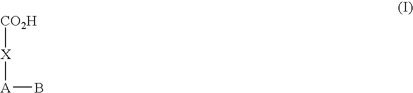
in which:
X is YCR¹R² or CR³=CR⁴;
A is aryl or heteroaryl, optionally su8bstituted by one or more substituents independently selected from hy6drogen, halogen, CN, OH, SH, nitro, S(O)ₙR⁵ (where n is 0, 1 or 2), OR⁵, NR⁶R⁷ or C₁₋₆alkyl, the latter group being optionally substituted by onje or more halogen atoms,
B is aryl or heteroaryl, optionally sub stituted by one or more substituents independently selected from from hydrogen, halogen, CN, OH, SH, nitro, CO₂R⁶, COR⁶, SO₂R⁸, OR⁸, SR⁸, SOR⁸, SO₂NR⁹R¹⁰, CONR⁹R¹⁰, NR⁹R¹⁰, NHSO₂R⁸, NR⁸SO₂R⁸, NR⁸CO₂R⁸, NHCOR⁸, NR⁸COR⁸, NR⁶CONR⁶R⁷, NR⁶SO₂NR⁶R⁷, aryl, heteroaryl, C₂-C₆ alkenyl, C₂-C₆ alkynyl, C₃-C₇ cycloalkyl or C₁₋₆alkyl, the latter four groups being optionally substituted by one or more substituents independently selected from halogen, C₃-C₇ cycloalkyl, OR⁶, NR⁶R⁷, S(O)ₙR⁵ (where n is 0, 1 or 2), CONR⁶R⁷, NR⁶COR⁷, SO₂NR⁶R⁷ and NR⁶SO₂R⁵;
X and B are attached to the the aryl or heteroary6l ring ortho relative to each other
Y is a bond, O, S(O)ₙ (where n is 0, 1 or 2), NR³ or CR¹R²;
R¹ and R² independently represent a hydrogen atom, halogen, C₂-C₆ alkenyl, C₂-C₆ alkynyl, C₃-C₇ cycloalkyl or a C₁₋₆alkyl group, the latter four groups being optionally substituted by one or more substituents independently selected from halogen, C₃-C₇ cycloalkyl, NR³R⁴, OR³, S(O)ₙR⁵ (where n is 0, 1 or 2); or
R¹ and R² together can form a 3-8 membered ring optionally containing one or more atoms selected from O, S, NR¹¹ and itself optionally substituted by one or more C₁-C₃ alkyl or halogen;
R³ and R⁴ independently represent hydrogen, or C₁₋₆alkyl
R⁵ is C₁₋₆alkyl or C₃-C₇ cycloalkyl, optionally substituted by one or more halogen atoms
R⁶ and R⁷ independently represents a hydrogen atom, C₁-C₆ alkyl or C₃-C₇ cycloalkyl, optionally substituted by one or more halogen atoms
R⁸ represents aryl, hereroaryl, C₃-C₇ cycloalkyl or C₁₋₆alkyl, the latter two groups may be optionally substituted by one or more substituents independently selected from halogen, C₃-C₇ cycloalkyl, aryl, heteroaryl OR⁶ and NR⁶R⁷, S(O)ₙR⁵ (where n=0,1 or 2, CONR⁶R⁷, NR⁶COR⁷, SO₂NR⁶R⁷ and NR⁶SO₂R⁵;
R⁹ and R¹⁰ independently represent aryl or heteroaryl, hy6drogen, C₃-C₇ cycloalkyl or C₁₋₆alkyl, the latter two groups beihng optionally substituted by one or more substituents independently selected from halogen, C₃-C₇ cycloalkyl, aryl, heteroaryl, OR⁶ and NR⁶R⁷, S(O)ₙR⁵ (where n=0, 1 or 2), CONR⁶R⁷, NR⁶COR⁷, SO₂NR⁶R⁷ and NR⁶SO₂R⁷; or
R⁹ and R¹⁰ together with the nitrogen atom to which they are attached can form a 3-8 membered saturated heterocyclic ring optionally containing one or more atoms selected from O, S(O)ₙ (where n=0, 1 or 2), NR¹¹, and itself optionally substituted by halogen or C₁-C₃ alkyl; and
R¹¹ represent a hydrogen atom, C₁₋₆alkyl, C₃-C₇ cycloalkyl, SO₂R⁵ or COC₁-C₄ alkyl, provided that:
When Y is O and A=phenyl, then B is not aryl or a six membered heterocyclic aromatic ring containing one ore more nitrogen atoms or a 6,6 or 6,5 fused bicycle containing one or more O, N, S atoms,
When Y is O and B is phenyl or a 6,6 or 6,5 fused bicycle containing one or more O, N, S atoms, then A is not aryl.

2. A compound according to claim 1 in which A is phenyl or a six membered heterocyclic aromatic ring containing one or more nitrogen atoms substituted in the para position to the acid with haologen, trifluoromethyl, cyano, amino or C₁₋₃alkyl.

3. A compound according to claim 1 in which A is phenyl or pyridyl substituted in thye para position to the acid with halogen, trifluoromethyl, cyano, amino or C₁₋₃alkyl.

4. A compound according to claim 1 in which X is CH₂CH₂, CH₂S, CH₂NH, CH₂NMe, CH₂O, CH₂, CH═CH or CHCH₃O.

5. A compound according to claim 1 in which X is CH₂CH₂, CH₂S, CH₂NH, CH₂NMe, CH₂, CH═CH.

6. A compound according to claim 1 in which B is phenyl, pyrazole, thienyl, furyl or indolyl, each optionally substituted as defined in claim 1.

7. A compound accordin to claim 1 selected from:
(4-Chloro-2-isoxazol-5-ylphenoxy)acetic acid
N-(5-Chlorobiphenyl-2-yl)glycine
3-(5-Chlorobiphenyl-2-yl)propanoic acid
(2E)-3-(5-Chlorobiphenyl-2-yl)acrylic acid
N-(5-Chlorobiphenyl-2-yl)-N-methylglycine
(5-Chlorobiphenyl-2-yl)acetic acid
{[5-Chloro-4′-(ethylthio)biphenyl-2-yl]thio}acetic acid
[5-Chloro-4′-(ethylsulfonyl)-2′-methylbiphenyl-2-yl]acetic acid
N-[4′-(Ethylsulfonyl)-5-(trifluoromethyl)biphenyl-2-yl]glycine
3-[4′-(Ethylsulfonyl)-2′-methyl-5-(trifluoromethyl)biphenyl-2-yl]propanoic acid
({2-[4-(Ethylsulfonyl)-2-methylphenyl]-6-methylpyridin-3-yl}oxy)acetic acid
[2-(2-Cyano-3-thienyl)-4-(trifluoromethyl)phenoxy]acetic acid
[2-(2-Furyl)-4-(trifluoromethyl)phenoxy]acetic acid
[2-(2-Chloro-3-thienyl)-4-(trifluoromethyl)phenoxy]acetic acid
[2-(2,5-Dichloro-3-thienyl)-4-(trifluoromethyl)phenoxy]acetic acid
[2-(2-Thienyl)-4-(trifluoromethyl)phenoxy]acetic acid
[2-(3-Thienyl)-4-(trifluoromethyl)phenoxy]acetic acid
[2-(5-Acetyl-2-thienyl)-4-(trifluoromethyl)phenoxy]acetic acid
[(5-Chloro-3′-cyanobiphenyl-2-yl)thio]acetic acid
(2S)-2-[2-[1-Methyl-3-(trifluoromethyl)-1H-pyrazol-5-yl]-4-(trifluoromethyl)phenoxy] propanoic acid
(2S)-2-[4-(Trifluoromethyl)-2-(1,3,5-trimethyl-1H-pyrazol-4-yl)phenoxy]propanoic acid
(2S)-2-[2-[1-Methyl-5-(trifluoromethyl)-1H-pyrazol-3-yl]-4-(trifluoromethyl)phenoxy] propanoic acid
(2S)-2-[2-{1-[(Dimethylamino)sulfonyl]-3-methyl-1H-pyrazol-4-yl} -4-(trifluoromethyl) phenoxy]propanoic acid
N-[4-Chloro-2-(5-methoxy-1H-indol-1-yl)phenyl]glycine
N-[4-Chloro-2-(5-cyano-1H-indol-1-yl)phenyl]glycine
({2-[2-Chloro-4-(methylsulfonyl)phenyl]-6-methylpyridin-3-yl}oxy)acetic acid
{[2-(3-Cyanophenyl)pyridin-3-yl]oxy}acetic acid
(2S)-2-({2-[2-Chloro-4-(methylsulfonyl)phenyl]-6-methylpyridin-3-yl}oxy)propanoic acid
{[6-Amino-2-(3-cyanophenyl)pyridin-3-yl]oxy}acetic acid
N-{4-Chloro-2-[5-(methylsulfonyl)-1H-indol-1-yl]phenyl}glycine,
3-[4′-(Methylsulfonyl)-3′,5-bis(trifluoromethyl)biphenyl-2-yl]propanoic acid
3-(5-Chloro-3′-cyanobiphenyl-2-yl)propanoic acid
3-[2′,5-Dichloro-4′-(methylsulfonyl)biphenyl-2-yl]propanoic acid
3-[3′-Fluoro-4′-(pyrrolidin-1-ylcarbonyl)-5-(trifluoromethyl)biphenyl-2-yl]propanoic acid
3-[2′-Chloro-4′-(methylsulfonyl)-5-(trifluoromethyl)biphenyl-2-yl]propanoic acid
3-[4′-(Ethylsulfonyl)-3′,5-bis(trifluoromethyl)biphenyl-2-yl]propanoic acid
3-[3′-Cyano-5′-fluoro-5-(trifluoromethyl)biphenyl-2-yl]propanoic acid
3-[3′-Cyano-5-(trifluoromethyl)biphenyl-2-yl]propanoic acid
3-[5-Chloro-3′-fluoro-4′-(phenylsulfonyl)biphenyl-2-yl]propanoic acid
3-[5-Chloro-4′-(pyridin-2-ylsulfonyl)biphenyl-2-yl]propanoic acid
and pharmaceutically acceptable salts thereof.

8. (canceled)

9. A method of treating a desease mediated by prostaglandin D2, which comprises administering to a patient a therapeutically effective amount of a compound of formula (I), or a pharmaceutically acceptable salt as defined in claim 1.

10. A method of treating a respiratory disease, in a patient suffering from or at risk of, said disease, which comprises administering to the patient a therapeutically effective amount of a compound of formula (I), or a pharmaceutically acceptable salt or solvate thereof, as defined in claim 1.

11. The method of claim 10, wherein the respiratory disease is asthma.

12. The method of claim 10, wherein the respiratory disease is rhinitis.
